(19) United States
(12) Patent Application Publication
(10) Pub. No.: US 2009/0022805 A1
(43) Pub. Date: Jan. 22, 2009

(54) Polypeptide microparticles having sustained release characteristics, methods and uses

(76) Inventors: Joram Slager, St. Louis Park, MN (US); Michael D. New, Eden Prairie, MN (US); John V. Wall, Woodbury, MN (US); Michael J. Burkstrand, Richfield, MN (US); Stephen J. Chudzik, (US); Pamela J. Reed, St. Paul, MN (US)

Correspondence Address: Kagan Binder, PLLC, 221 Main Street North, Suite 200, Stillwater, MN, 55082, US

(21) Appl. No.: 12/215,508

(22) Filed: Jun. 27, 2008

Related U.S. Application Data
(60) Provisional application No. 60/937,682, filed on Jun. 28, 2007.

(51) Int. Cl. A61K 9/14; A61P 43/00

(52) U.S. Cl. 424/486

(57) ABSTRACT

The invention provides polypeptide microparticles having control release features, particular methods for the preparation of such microparticles, and drug delivery systems that include polypeptide microparticles.

### CROSS-REFERENCE TO RELATED APPLICATION

This application claims the benefit of U.S. Provisional Patent Application Ser. No. 60/937,682, filed Jun. 28, 2007, entitled POLYPEPTIDE MICROPARTICLES HAVING SUSTAINED RELEASE CHARACTERISTICS, METHODS AND USES, the disclosure of which is incorporated herein by reference.

### TECHNICAL FIELD

The present invention relates to polypeptide microparticles and methods for their formation. The polypeptide microparticles are suitable for providing controlled, sustained release of polypeptide. The present invention also relates to methods and systems using polypeptide microparticles for therapeutic uses.

### BACKGROUND

Therapeutic agents can be introduced into a subject by several different routes. Most commonly, therapeutic agents are orally administered because it is a convenient, safe, and cost effective way to making the agent systemically available to the body. However, in many cases, oral administration is not preferred. For example, certain therapeutic agents are either not stable in, or adequately taken into the body, by the digestive tract. Therapeutic agents such as proteins, polypeptides, or oligopeptides (collectively referred to herein as “polypeptides”) are typically not orally administered.

Therapeutic polypeptides are typically administered by routes that avoid conditions that destroy the polypeptide, such as would occur with proteolysis in portions of the digestive tract. Commonly used injection routes for polypeptides include subcutaneous, intramuscular, and intravenous injections. Frequent injections are often necessary due to short plasma half-lifes of polypeptides. In some cases, mucosal administration of polypeptides can be performed using methods that place the polypeptide in contact with membranes lining the urogenital or and respiratory tracts.

Many current therapeutic preparations of polypeptide therapeutics are liquid formulations (for example, liquid formulations of insulin), which are injected into a subject to provide a therapeutic effect. However, many of these injectable compositions provide a therapeutic response over a limited period of time.

Solid formulations of polypeptides have been prepared in attempt to lengthen the therapeutic window for polypeptide. One approach is to crush or grind lyophilized polypeptides into small particulates, which can be administered to a patient. This approach is less than desirable, as it can be detrimental to the activity of the polypeptide.

Another approach for delivering polypeptides to a subject is to use polymer microparticles that are associated with polypeptides. Microparticles refer to those particles having a diameter of less than 1 mm, and are more typically found as having a diameter of less than 0.1 mm (100 μm), and includes those in the upper nanometer range, such as about 100 nm or greater. Most microparticles are spherical in shape (i.e., microspheres), although microparticles may be observed having other non-spherical shapes. Spray drying, phase separation, solvent evaporation, and emulsification are common techniques used to make microparticles, which are typically formed from synthetic or natural polymers. However, many microparticle preparations have low polypeptide content due the presence of a larger content of excipient polymer in the microparticle. This can significantly limit the amount of polypeptide that can become available to a subject upon administration of the microparticles.

Further, challenges relate to the controlling release of the polypeptide from microparticles. For example, it may be desirable to limit and/or substantially eliminate an initial “burst” of a high concentration of the polypeptide. This may be particularly desirable when it is desired to provide a sustained release (e.g., over weeks or months) of the polypeptide from the microparticles. Thus, it may be desirable to modulate release of the polypeptide from the microparticles to provide a release profile that is within a therapeutic window and can last for the duration of a treatment course.

### SUMMARY OF THE INVENTION

The present invention is directed to polypeptide microparticles, particular methods for the preparation of such microparticles, and drug delivery systems that include polypeptide microparticles. In some aspects, the polypeptide microparticles can be used for the treatment of a medical condition in a subject, in which polypeptides are released from the microparticles in a controlled, sustained manner, and provide a therapeutic effect to a subject. The polypeptide microparticles can also be used in association with a drug delivery system that is implanted or formed at a target location in the body.

In many aspects, the polypeptide microparticles can be placed within the body where they dissolve and polypeptide is released, providing a therapeutic effect to a subject. The microparticles can be introduced into the body alone, or in combination with another component that can contribute to modulating release of the polypeptides. The microparticles can be used in therapies so the polypeptide exerts a site-specific effect, or alternatively, a more general systemic therapeutic effect throughout the body.

The microparticles can also be used in conjunction with a drug delivery device. The microparticles can be associated with the device, in a manner that they are releasable from the device, immobilized on or within the device, or both. For example, the polypeptide microparticles can be present in a polymeric matrix forming a coating, the coating being associated with a portion of an implantable medical device.

Generally, the invention provides polypeptide microparticles that include one or more components that modulate release of the polypeptide from the particle. The studies associated with the invention have shown that the microparticles can be used to control release of various polypeptides, and in particular antibodies and antibody fragments, such as Fab and Fab′2 fragments.

In some aspects, the invention provides polypeptide microparticles that comprise a core of predominantly polypeptide and a coating on the polypeptide core that controls release of the polypeptide.

In a first aspect, the invention provides a microparticle comprising a core formed predominantly of polypeptide, and a microparticle coating on the core. The microparticle coating on the core includes a crosslinked polymeric matrix, which can be formed by reacting reactive groups pendent from the polymer to form the matrix. The polypeptide is capable of being released from the microparticle and the microparticle coating is able to modulate polypeptide release. The crosslinked polymeric matrix can include degradable or non-degradable polymeric material.

In some preferred aspects, the microparticle includes one or more of the following additional feature(s): polypeptide in an amount of 50% wt or greater, or 70% wt or greater in the core; a core to microparticle coating weight ratio in the range of 96:4 to 50:50; polymerized groups pendent from polymers forming the polymeric matrix, such as reacted ethylenically unsaturated (e.g., vinyl) groups; a polymerization initiator proximal to the core; and/or polymers forming the polymeric matrix having a molecular weight in the range of 1,000 Da to 500,000 Da.

In one aspect, the microparticles comprise a core comprising predominantly polypeptide and a microparticle coating in contact with the core. The microparticle coating comprises a crosslinked matrix of biodegradable polysaccharide. In some preferred aspects, the crosslinked biodegradable polysaccharide microparticle coating includes one or more of the following additional feature(s): a matrix formed of a biodegradable polysaccharide having a molecular weight in the range of about 1000 Da to about 500,000 Da; and/or a biodegradable polysaccharide selected from the group consisting of maltodextrin, amylose, and polyalditol.

The use of natural biodegradable polysaccharides, such as amylose, maltodextrin, or polyalditol, provides advantages for use as a component in the microparticles. These advantages include resistance to matrix breakdown from hydrolytic degradation, improved biocompatibility because the natural biodegradable polysaccharides can be obtained from non-animal (plant) sources, and lack of acidic degradation products (as otherwise would be found in polyglycolide-type polymeric materials). As such there is minimal or no immunogenic, inflammatory, or toxic risk when the microparticles are used in vivo.

For example, microparticles having a natural biodegradable polysaccharide-based coating can be manipulated in a non-biological, aqueous-based-medium without risk that the coating will prematurely degrade due to non-enzyme-meditated hydrolysis. Coatings that are based on biodegradable polymers such as poly(lactide) or poly(lactide-co-glycolide) are subject to hydrolysis even at relatively neutral pH ranges (e.g., pH 6.5 to 7.5) and therefore do not offer this advantage. The microparticles coatings of the invention can provide stability in the presence of an aqueous environment. A semi-stable or stable microparticle coating can be formed which allows the polypeptide microparticles to be manipulated in a composition that would otherwise dissolve the polypeptide microparticles if the coating were not present. Some of these compositions may be used to prepare a polymeric matrix, such as one for device coating. Therefore, the microparticle coating can facilitate preparation of polypeptide microparticle-containing polymeric matrices, such as device coatings.

As an alternative to a degradable polymeric material, the coating formed on the microparticle core can include a non-degradable polymer. In one aspect, the invention provides microparticles comprising a core comprising a polypeptide, and a polypeptide release controlling coating in contact with the core, the coating comprising a crosslinked polymeric matrix formed of a polymer, wherein the polymer comprises monomer or monomers including uncharged polar moieties, and one or more pendant reactive groups. For example, the polymer can comprise N,N-disubstituted acrylamide. In other aspects, the polymer can comprise polyethylene glycol. The reactive groups pendent from the polymer can be thermochemically reactive groups, photochemically reactive groups, or a combination thereof.

The invention also provides a method for forming a microparticle comprising a core of predominantly polypeptide and a microparticle coating comprising a crosslinked polymeric matrix. The method includes a step of providing a core particle comprising predominantly polypeptide in a liquid composition. In another step the core particle is mixed with a first component comprising a first reactive group. In another step the core particle is mixed with a second component comprising a polymer and a pendent a second reactive group; wherein either: (i) the first reactive group is reactive with the second reactive group, thereby forming the crosslinked polymeric matrix, or (ii) the first reactive group comprises a polymerization initiator group and the second reactive group comprises a polymerizable group. In the case feature (ii) is used the method additionally comprises a step of activating the initiator group to cause polymerization of the first component, thereby forming the crosslinked polymeric matrix. The step of mixing with a first component comprising a first reactive group can be performed before, after, or at the same time as the step of mixing with the second component.

In some preferred aspects, the method includes one or more of the following additional steps or feature(s): core particle present in the composition at a concentration in the range of 4 mg/mL to 50 mg/mL; mixing the first component with the core particle at a weight ratio in the range of 0.5:100 to 10:100; an additional step of adding a phase separation agent to the liquid composition, wherein the phase separation agent comprises an amphiphilic compound; adding the phase separation agent at concentration in the range of 100 mg/mL to 500 mg/mL; and/or adding the phase separation agent at a temperature in the range of 20° C. to 55° C.

The invention also provides another method for forming a microparticle comprising a core of predominantly polypeptide and a microparticle coating comprising a crosslinked polymeric matrix. However, this method does not require initially providing a core particle. Rather, a nucleation step is carried out to cause formation of the polypeptide core in an initial step of the process. The method includes a step of providing a liquid composition comprising polypeptide, nucleating agent, and polymer comprising pendent reactive groups. Another step includes heating the composition to a temperature above room temperature. Another step includes adding a phase separation agent comprising an amphiphilic compound to the composition. Another step includes cooling the composition comprising the amphiphilic compound. Another step includes extracting at least a portion of the phase separation agent. Another step includes activating the pendent reactive groups to crosslink the polymer to form the crosslinked polymeric matrix.

In some preferred aspects, the method includes one or more of the following additional steps or feature(s): polypeptide being present in the composition at a concentration in the range of 10 mg/mL to 50 mg/mL; nucleating agent being present in the composition at a concentration in the range of 1 μg/mL to 10 μg/mL; the polymer comprising pendent reactive groups being present in the composition at a concentration in the range of 1 mg/mL to 30 mg/mL; heating the composition to a temperature (above room temperature) in the range of 30° C. to 70° C.; providing phase separation agent in the composition at a concentration in the range of 100 mg/mL to 500 mg/mL; and/or cooling the composition having the amphiphilic compound to a temperature in the range of −20° C. to 4° C.

While in some aspects the invention provides polypeptide microparticles that comprise a core of predominantly polypeptide and a coating on the polypeptide core, in other aspects the coating includes a non-crosslinked polymeric material that adheres to the core and controls release of the polypeptide. The coated microparticles are easily prepared and provide excellent polypeptide release control, such as when incorporated into a polymeric matrix that forms a coating on the surface of an implantable medical article.

Therefore, in another aspect, the invention provides a microparticle comprising a core formed predominantly of polypeptide and a microparticle coating comprising a non-crosslinked polymeric layer that includes a polymer comprising pendent hydrophobic groups. The polypeptide is capable of being released from the microparticle and the microparticle coating is able to modulate release of the polypeptide from the microparticle.

In some cases the polypeptides in the core of the microparticle comprise an antibody or an antibody fragment, such as Fab or Fab′2 fragment.

In some preferred aspects, the microparticle includes one or more of the following additional feature(s): polypeptide in an amount of 50% wt or greater, or 70% wt or greater in the core; the polymer comprising pendent hydrophobic groups also comprising a backbone comprising monomer or monomers including uncharged polar moieties; the polymer comprising pendent hydrophobic groups also comprising a poly(ethyleneimine) backbone; the polymer comprising pendent hydrophobic groups further comprising pendent quaternary amine groups; a weight ratio of the polymer backbone to the pendent hydrophobic groups in the range of about 1:0.43 to about 1:1.28, about 1:0.64 to about 1:1.06, or about 1:0.85; the polymer comprising pendent hydrophobic groups having a molecular weight of 250,000 Da or less; and/or a weight ratio of the core to the microparticle coating in the range of 100:0.5 to 100:5.

The microparticles can be formed by a method comprising the steps of providing a core particle comprising predominantly polypeptide in a liquid composition, and mixing the core particle with a polymer comprising pendent hydrophobic groups

In some preferred aspects, the method includes one or more of the following additional step(s) or feature(s): mixing the polymer comprising pendent hydrophobic groups with the core particle at a weight ratio in the range of 100:0.5 to 100:5 and/or using a composition comprising a halogenated solvent.

In other aspects, the microparticle coating is an optional feature, and the microparticle comprises polypeptide that is incorporated in a crosslinked biodegradable polymeric matrix, wherein the crosslinked polymeric matrix of the microparticle itself controls release of the polypeptide. The polypeptide is at least substantially homogeneously mixed in the biodegradable polymer matrix in the microparticle. Accordingly, the invention generally provides polypeptide microparticles that are formed of a crosslinked matrix of biodegradable polysaccharide. In these aspects, a component of the microparticle itself (the degradable polysaccharide used to form the microparticle) controls release of the polypeptide.

Thus, in some embodiments, the invention provides microparticles comprising a crosslinked matrix of biodegradable polysaccharide, and a polypeptide incorporated in the crosslinked matrix, wherein the biodegradable polysaccharide has a molecular weight of 500,000 Da or less, wherein the microparticle comprises a ratio of polypeptide to biodegradable polysaccharide in the range of 3:1 to 1:3 by weight, and wherein the crosslinked matrix comprises polymerized groups that covalently couple biodegradable polysaccharide together.

In some cases the polypeptides comprise an antibody or an antibody fragment, such as a Fab or Fab′2 fragment.

In some preferred aspects, the microparticle includes one or more of the following additional feature(s): a biodegradable polysaccharide selected from the group consisting of maltodextrin, amylose, and polyalditol, or a combination thereof; a polypeptide:maltodextrin ratio of 2:1; polymerized groups comprising reacted methacrylate groups; polymerized groups pendent from the biodegradable polysaccharide in an amount in the range of DS 0.1 to DS 0.5; and/or a biodegradable polysaccharide having a molecular weight in the range of 1,000 Da to 100,000 Da.

The invention also provides a method for preparing such a microparticle. The method comprises a step of providing a liquid composition comprising (i) polypeptide and (ii) biodegradable polysaccharide having a molecular weight of 500,000 Da or less, and comprising pendent polymerizable groups. Another step includes adding a phase separation agent to the composition. Another step includes adding a polymerization initiator to the composition. Another step includes cooling the composition. Another step includes activating the initiator to couple the biodegradable polysaccharides, thereby forming microparticles comprising a crosslinked matrix of biodegradable polysaccharide and polypeptide in the crosslinked matrix.

In some preferred aspects, the method includes one or more of the following additional step(s) or feature(s): polypeptide present in the liquid composition at a concentration in the range of 10 mg/mL to 40 mg/mL; biodegradable polysaccharide present in the composition at a concentration in the range of 1 mg/mL to 120 mg/mL; performing the steps of adding a phase separation agent and a polymerization initiator simultaneously (e.g., the phase separation agent and the polymerization initiator are present in the same composition); a polymerization initiator selected from a photoinitiator and a redox initiator; and/or the phase separation agent being present in the composition at a concentration in the range of 100 mg/mL to 500 mg/mL.

In still further aspects, a component separate from the microparticles themselves can assist in modulating release of polypeptide from the microparticles. In these embodiments, the microparticles can be used in conjunction with a separate component that includes a polymer system, which can assist in modulating release of the polypeptide. The polymer system is used in the form of a polymeric matrix. In some embodiments, the polypeptide is released from the microparticles and eluted from the matrix in what is herein referred to as an “elution control matrix.” The elution control matrix has been shown to provide excellent control over polypeptide release when using the microparticles of the invention, and is particularly suitable for the in vivo release of polypeptide over prolonged treatment periods. Any of the polypeptide microparticles of the invention, coated or uncoated, can be used in associated with the elution control matrix for controlled release of the polypeptide. The elution control matrix can include biostable or biodegradable components.

In some cases the polypeptide microparticles are immobilized in a polymeric matrix that is associated with an implantable medical device (such as in a coating on a surface of the device). In some cases, the microparticles can be included in a polymer system that is utilized to fabricate a medical device or an implantable medical article, such as a drug delivery filament. For example, the microparticles of the invention can be immobilized in a biodegradable polymeric matrix which can be formed into a suitable shape for implantation at a target location in the body.

In some aspects, the invention provides an elution control matrix for the controlled release of a polypeptide. The elution control matrix comprises a polymeric matrix and polypeptide microparticles within the polymeric matrix. In some cases, the polypeptide microparticles within the polymeric matrix comprise a core formed predominantly of polypeptide, and a microparticle coating on the core, wherein the microparticle coating on the core includes a crosslinked polymeric matrix. In some cases the polypeptide microparticles within the polymeric matrix comprise (i) a crosslinked matrix of biodegradable polysaccharide, and (ii) polypeptide in the crosslinked matrix, wherein the biodegradable polysaccharide has a molecular weight of 500,000 Da or less, wherein the microparticle comprises a ratio of polypeptide to biodegradable polysaccharide in the range of 3:1 to 1:3 by weight, and wherein the crosslinked matrix comprises reacted polymerizable groups that covalently couple biodegradable polysaccharide together. In some cases the polypeptide microparticles comprise predominantly polypeptide and a microparticle coating, the microparticle coating comprising a non-crosslinked polymeric layer including a polymer having pendent hydrophobic groups.

In some cases the polypeptides microparticles in the elution control matrix comprise an antibody or an antibody fragment, such as a Fab or a Fab′2 fragment.

In some cases the polymeric matrix of the elution control matrix comprises one or more of the following polymers: poly(n-butyl methacrylate), a polyethylene glycol block copolymer, and/or poly(ethylene-co-vinyl acetate).

In some aspects the microparticles are present in the matrix in an amount in the range of 30% to 70% by weight solids.

In some aspects the elution control matrix is in the form of a coating on an implantable medical device. An exemplary medical device having an elution control matrix coating is an implantable ophthalmic device, such as one that can deliver polypeptide to the vitreal chamber in the eye.

In still further aspects, the invention provides methods for treating medical conditions using the elution control matrix. Types of medical conditions include those that benefiting from the administration of a polypeptide-based therapeutic agent in a subject.

### BRIEF DESCRIPTION OF THE FIGURES

FIG. 1 is a graph showing cumulative Fab release (%) from microparticle coated intravitreal implants.

FIG. 2 is a graph showing cumulative Fab release (%) from microparticle coated intravitreal implants.

FIG. 3 is a graph showing cumulative Fab release (%) from microparticles.

FIG. 4 is a graph showing cumulative Fab release (%) from microparticles.

FIG. 5 is a graph showing cumulative Fab release (%) from microparticles.

FIG. 6 is a graph showing cumulative Fab release (%) from microparticle coated intravitreal implants.

FIG. 7 is a graph showing cumulative IgG release (%) from microparticle coated intravitreal implants.

FIG. 8 is a graph showing cumulative IgG release (%) from microparticle coated intravitreal implants.

FIG. 9 is a graph showing cumulative Fab release (%) from microparticles.

FIG. 10 is a graph showing cumulative Fab release (%) from microparticles.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

In one aspect, the invention provides a microparticle comprising a core comprising a polypeptide, wherein the polypeptide is the predominant component of the core, and a polypeptide release controlling microparticle coating in contact with the core, the coating comprising a crosslinked polymer matrix. In some cases the crosslinked polymer matrix comprises crosslinked biodegradable polysaccharides.

The expression “microparticles” is used herein as a general term for particles of a certain size according to the art that is known per se. One type of microparticle is therefore constituted by microspheres, which have a substantially spherical form, whilst the term microparticle can in general include deviation from such a perfect spherical form. A spherical polypeptide microparticle will have, from a center of the polypeptide microparticle, the distance from the center to the outer surface of the microparticle is about the same for any point on the surface of the microparticle. A substantially spherical microparticle is where there may be a difference in radii, but the difference between the smallest radii and the largest radii is generally not greater than about 40% of the smaller radii, and more typically less than about 30%, or less than 20%. The term microcapsule, which is known per se, also falls within the expression “microparticle” according to the prior art. Generally, microparticles are solid or semi-solid particles. Microparticles have been utilized in many different applications, primarily separations, diagnostics, and drug delivery.

The microparticles may be administered to a human or animal, for example, by oral or parenteral administration, including intravenous, subcutaneous or intramuscular injection; administration by inhalation; intraarticular administration; mucosal administration; ophthalmic administration; and topical administration. Intravenous administration includes catheterization or angioplasty. Administration may be for purposes such as therapeutic and diagnostic purposes as discussed herein.

According to the invention, microparticles are fabricated to provide controlled release of polypeptide therefrom. For ease of discussion, reference will repeatedly be made to a “polypeptide.” While reference will be made to a “polypeptide,” it will be understood that the invention can provide any number of polypeptides to a treatment site. Thus, reference to the singular form of “polypeptide” is intended to encompass the plural form as well.

As used herein, a polypeptide refers to an oligomer or polymer including two or more amino acid residues, and is intended to encompass compounds referred to in the art as proteins, polypeptides, oligopeptides, peptides, and the like. More, specific classes of peptides include enzymatic polypeptides (enzymes), antibodies, antibody fragments, neuropeptides, and peptide hormones. The twenty, common, naturally-occurring amino acids residues and their respective one-letter symbols are as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine).

The polypeptides can also include one or more rare and/or non-natural amino acids. Naturally-occurring, rare amino acids include selenocysteine (Sec) and pyrrolysine (Pyl). Non-natural amino acids are typically organic compounds having a similar structure and reactivity to that of naturally-occurring amino acid counterpart. Non-natural amino acids include, for example, cyclic amino acid analogs, amino alcohols, D-amino acids, propargylglycine derivatives, beta amino acids, gamma amino acids, 2-amino-4-cyanobutyric acid derivatives, and Weinreb amides of α-amino acids. Incorporation of such amino acids into a polypeptide may serve to increase the stability, reactivity and/or solubility of the polypeptide

Polypeptides of the invention can also include those that are modified with, or conjugated to, another biomolecule or biocompatible compound. For example, the polypeptide can be a peptide-nucleic acid (PNA) conjugate, polysaccharide-peptide conjugates (e.g., glycosylated polypeptides; glycoproteins), a poly(ethyleneglycol)-polypeptide conjugate (PEG-ylated polypeptides).

In some modes of practice, the microparticles are prepared from polypeptides having a molecular weight of about 10,000 Da or greater, or about 20,000 Da or greater; more specifically in the range of about 10,000 Da to about 100,000 Da, or in the range of about 25,000 Da to about 75,000 Da.

One class of polypeptides that can be associated with the microparticles of the invention includes antibodies and antibody fragments. Antibodies (immunoglobulins) are large glycoproteins (typically of about 100,000 Da or greater) containing antigen binding regions and have an overall “Y” shape. The polypeptides can be glycosylated, since antibody polysaccharide chains are typically attached to amino acid residues by N-linked glycosylation and occasionally by O-linked glycosylation.

The polypeptides can also include a disulfide bond; an antibody consists of two identical heavy chains and two identical light chains that are connected by disulfide bonds. Each heavy chain has two regions, known as the constant and variable regions. The polypeptides can also include an immunoglobulin domain; the variable domain of any heavy chain is composed of a single immunoglobulin domain which is about 110 amino acids long. A light chain has two successive domains: one constant domain and one variable domain. The approximate length of a light chain is 211 to 217 amino acids. The polypeptide can also include a peptide sequence capable of affinity interaction with a ligand; the variable regions of the heavy and light chains provide antigen/epitope binding specificity.

This portion of the antibody region is called the Fab fragment, antigen binding) region of the antibody and is composed of one constant and one variable domain from each heavy and light chain of the antibody. The paratope is shaped at the amino terminal end of the antibody monomer by the variable domains from the heavy and light chains.

Antibody light and heavy chains are composed of structural domains called immunoglobulin (Ig) domains. These domains contain about 70-110 amino acids and are classified into different categories (for example, variable or IgV, and constant or IgC) according to their size and function. They possess a characteristic immunoglobulin fold in which two beta sheets create a “sandwich” shape, held together by interactions between conserved cysteines and other charged amino acids.

A variety of antibody and antibody fragments are commercially available, obtainable from deposited samples, or can be prepared by techniques known in the art.

Monoclonal antibodies (mAbs) can be obtained by any technique that provides for the production of antibody molecules by continuous cell lines in culture. These include, for example, the hybridoma technique (Kohler and Milstein, Nature, 256:495-497 (1975)); the human B-cell hybridoma technique (Kosbor et al., Immunology Today, 4:72 (1983); and the EBV-hybridoma technique (Cole et al., Monoclonal Antibodies and Cancer Therapy, Alan R. Liss, Inc., pp. 77-96 (1985)). Such antibodies may be of any immunoglobulin class including IgG, IgM, IgE, IgA, IgD and any subclass thereof.

Fab or Fab′2 fragments can be generated from monoclonal antibodies by standard techniques involving papain or pepsin digestion, respectively. Kits for the generation of Fab or Fab′2 fragments are commercially available from, for example, Pierce Chemical (Rockford, Ill.).

Examples of antibodies and antibody fragments that can be used in connection with the microparticles of the present invention include, but are not limited to, therapeutic antibodies including trastuzumab (Herceptin™), a humanized anti-HER2 monoclonal antibody (mAb); alemtuzumab (Campath™), a humanized anti-CD52 mAb; gemtuzumab (Mylotarg™), a humanized anti-CD33 mAb; rituximab (Rituxan™), a chimeric anti-CD20 mAb; ibritumomab (Zevalin™), a murine mAb conjugated to a beta-emitting radioisotope; tositumomab (Bexxar™), a murine anti-CD20 mAb; edrecolomab (Panorex™), a murine anti-epithelial cell adhesion molecule mAb; cetuximab (Erbitux™), a chimeric anti-EGFR mAb; bevacizumab (Avastin™), a humanized anti-VEGF mAb; Ranibizumab (Leucentis™), an anti-vascular endothelial growth factor mAb fragment; satumomab (OncoScint™) an anti-pancarcinoma antigen (Tag-72) mAb; pertuzumab (Omnitarg™) an anti-HER2 mAb; and daclizumab (Zenapax™) an anti IL-2 receptor mAb.

The polypeptide can also be selected from cell response modifiers. Cell response modifiers include chemotactic factors such as platelet-derived growth factor (PDGF), neutrophil-activating protein, human pigment-epithelium derived growth factor (PEDF), monocyte chemoattractant protein, macrophage-inflammatory protein, SIS (small inducible secreted) proteins, platelet factor, platelet basic protein, melanoma growth stimulating activity, epidermal growth factor, transforming growth factor (alpha), fibroblast growth factor, platelet-derived endothelial cell growth factor, insulin-like growth factor, nerve growth factor, vascular endothelial growth factor, bone morphogenic proteins, and bone growth/cartilage-inducing factor (alpha and beta). Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins.

The polypeptide can also be selected from therapeutic enzymes, such as proteases, phospholipases, lipases, glycosidases, cholesterol esterases, and nucleases. Specific examples include recombinant human tissue plasminogen activator (alteplase), RNaseA, RNaseU, chondroitinase, pegaspargase, arginine deaminase, vibriolysin, sarcosidase, N-acetylgalactosamine-4-sulfatase, glucocerebrocidase, α-galactosidase, and laronidase.

Although not limited to such, the microparticles of the invention are particularly useful for delivering therapeutic materials that are large hydrophilic molecules, such as polypeptides (including proteins and peptides), nucleic acids (including DNA and RNA), polysaccharides (including heparin), as well as particles, such as viral particles, and cells. In one aspect, the polypeptide has a molecular weight of about 10,000 or greater, or about 20,000 Da or greater; more specifically in the range of about 10,000 Da to about 100,000 Da, or in the range of about 25,000 Da to about 75,000 Da.

The particular polypeptide, or combination of polypeptides, can be selected depending upon one or more of the following factors: the application of the microparticles, the medical condition to be treated, the anticipated duration of treatment, characteristics of the implantation site, the number and type of polypeptides to be utilized, and the like.

Generally, the invention relates to the ability to control release of polypeptides from microparticles. This is accomplished by providing one or more polymeric components in association with the microparticles, wherein the polymeric component(s) modulate release of the polypeptide from the microparticle. In some aspects, the polymeric component that modulates release is included in the microparticle itself. In other aspects, the polymeric component that modulates release can be included as a coating on a microparticle core, the core including the polypeptide to be released. In still further aspects, the polymeric component that modulates release can be a polymeric matrix in which the microparticles are contained. In accordance with these latter aspects, the polymeric matrix can be, for example, a coating on a surface of a medical article or could be utilized to fabricate the body of the medical article itself. Each of these aspects will be described.

In some aspects the microparticle comprises a core comprising polypeptide, and a polypeptide release controlling coating in contact with the core.

The “core” in these aspects is a polypeptide microparticle. In some aspects, then, the inventive concepts can be utilized with virtually any microparticle that includes a polypeptide, wherein it is desirable to control release of the polypeptide from the microparticle. As such, the term “core” is understood to encompass microparticles containing polypeptide, regardless of the method by which such microparticles are formed, so long as the coating compositions described herein can be associated with these microparticles.

In preferred aspects the microparticle core is formed predominantly from polypeptide. This allows the amount of polypeptide that is released from the microparticle to be maximized, providing a high amount of therapeutic agent per amount of material that is introduced into the body.

In some aspects, the polypeptide microparticles can be formed as described in commonly owned patent application entitled “Polypeptide Microparticles,” Slager et al., U.S. Ser. No. 60/937,492, filed Jun. 28, 2007. Generally, these microparticles are formed in a solution, by coalescing polypeptides with a nucleating agent to form polypeptide nuclei; mixing a phase separation agent with the solution to further coalesce polypeptide around the polypeptide nuclei, thereby forming a mixture; cooling the mixture to form polypeptide microparticles; and removing all or part of the phase separation agent from the polypeptide microparticles. Using this method, the formed polypeptide “core” can have an amount of polypeptide, by weight, of about 90% or greater, such as in the range of about 90% to about 99.99%, of about 95% or greater, such as in the range of about 95% to about 99.99%, of about 97.5% or greater, such as in the range of about 97.5% to about 99.99%, of about 99% or greater, such as in the range of about 99% to about 99.99%, of about 99.5% or greater, such as in the range of about 99.5% to about 99.99%.

In some embodiments the invention provides polypeptide microparticles that include a (i) a core comprising predominantly polypeptide; and (ii) a microparticle coating, wherein the coating can be formed from polymers that are crosslinked together, or the coating can be formed from polymers that are not crosslinked together. In either case the “core”-“coating” arrangement of these microparticles can include microparticles having structures wherein: (a) the core material(s) (polypeptide) are substantially or entirely separated from the coating material(s) (polymer); or where the (b) the core material(s) (polypeptide) are partially blended with the coating material(s) (polymer).

One exemplary microparticle structure of the invention has a polypeptide core and a polymeric coating on the polypeptide core, and which is typical of many “core”-“shell” types of microparticle structures. In these structures there is substantially little, or no, polymeric material (of the coating) in the polypeptide core, and substantially little, or no, polypeptide in the polymeric coating.

Another exemplary microparticle of the invention has polypeptide core, a polymeric coating, and an interfacial zone of blended polypeptide and polymer between the coating and the core. In these structures a distinct border between the core and the coating is blurred by the interfacial zone. In some modes of practice, during the process of forming the coating on the polypeptide core, mixing of the coating polymer and the polypeptide of the core can occur thereby creating the interfacial zone. A gradient is thought to exist in the interfacial zone, with the concentration of coating polymer greater near the coating, and the concentration of the polypeptide greater near the core. It is thought that the mixing of the coating polymer and the polypeptide of the core may occur by solubilization of a small amount of polypeptide during the coating process and/or diffusion of the coating polymer into the particle core. Nonetheless, such a microparticle structure having a greater concentration of coating polymer near the outer surface of the microparticle, and a greater concentration of polypeptide near the center of the microparticle, is understood to fall within the scope of a “core”-“coating” arrangement of the present invention.

Although the microparticle coating process may begin with a polypeptide “core” particle with a very high weight percentage of polypeptide (for example, of about 90% wt or greater, such as prepared by Slager et al., supra), the amount of polypeptide in the core of the coated microparticle can be lower, such as greater than 50% wt, or about 70% wt or greater.

Degradable or non-degradable polymers, or combinations thereof, can be used to form the coating on a microparticle core, wherein the polymers are crosslinked. One class of degradable polymers are natural biodegradable polysaccharides.

In other embodiments of the invention, a “core”-“coating” structure is not a required feature of the microparticle, but rather, the polypeptide microparticles have a crosslinked matrix of natural biodegradable polysaccharide throughout at least the center of the microparticle, with polypeptide incorporated in the matrix. Natural biodegradable polysaccharide having pendent groups which can crosslink the polysaccharides, such as polymerizable groups, groups, and initiator systems as described herein can be used in methods for forming these microparticles.

As referred to herein, a “natural biodegradable polysaccharide” refers to a non-synthetic polysaccharide that is capable of being enzymatically degraded but that is generally non-enzymatically hydrolytically stable. Natural biodegradable polysaccharides include polysaccharide and/or polysaccharide derivatives that are obtained from natural sources, such as plants or animals. Natural biodegradable polysaccharides include any polysaccharide that has been processed or modified from a natural biodegradable polysaccharide (for example, maltodextrin is a natural biodegradable polysaccharide that is processed from starch). Exemplary natural biodegradable polysaccharides include hyaluronic acid, starch, dextran, heparin, chondroitin sulfate, dermatan sulfate, heparan sulfate, keratan sulfate, dextran sulfate, pentosan polysulfate, and chitosan. Preferred polysaccharides are low molecular weight polymers that have little or no branching, such as those that are derived from and/or found in starch preparations, for example, amylose and maltodextrin. Therefore, the natural biodegradable polysaccharide can be a substantially non-branched or non-branched poly(glucopyranose) polymer.

Because of the particular utility of the amylose and maltodextrin polymers, it is preferred that natural biodegradable polysaccharides in accordance with the invention have an average molecular weight of 500,000 Da or less, 250,000 Da or less, 100,000 Da or less, or 50,000 Da or less. It is also preferred that the natural biodegradable polysaccharides have an average molecular weight of 500 Da or greater. A particularly preferred size range for the natural biodegradable polysaccharides is in the range of about 1000 Da to about 100,000 Da. Natural biodegradable polysaccharides of particular molecular weights can be obtained commercially or can be prepared. The decision of using natural biodegradable polysaccharides of a particular size range may depend on factors such as the physical characteristics of the biodegradable composition (e.g., viscosity), the desired rate of degradation of the medical article, the presence of other optional moieties in the biodegradable composition, for example, polypeptides, and the like.

As used herein, “amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4% to 5% of the total linkages. Generally amylopectin derived from plant sources has a molecular weight of 1×10⁷ Da or greater.

Amylose can be obtained from, or is present in, a variety of sources. Typically, amylose is obtained from non-animal sources, such as plant sources. In some aspects, a purified preparation of amylose is used as starting material for the preparation of the amylose polymer having coupling groups. In other aspects, as starting material, amylose can be used in a mixture that includes other polysaccharides.

For example, in some aspects, starch preparations having a high amylose content, purified amylose, synthetically prepared amylose, or enriched amylose preparations can be used in the preparation of amylose having the coupling groups. In starch sources, amylose is typically present along with amylopectin, which is a branched polysaccharide. According to the invention, it is preferred to use coating compositions that include amylose, wherein the amylose is present in the composition in an amount greater than amylopectin, if present in the composition. For example, in some aspects, starch preparations having high amylose content, purified amylose, synthetically prepared amylose, or enriched amylose preparations can be used in the preparation of amylose polymer having the coupling groups. In some embodiments the composition includes a mixture of polysaccharides including amylose wherein the amylose content in the mixture of polysaccharides is 50% or greater, 60% or greater, 70% or greater, 80% or greater, or 85% or greater by weight. In other embodiments the composition includes a mixture of polysaccharides including amylose and amylopectin and wherein the amylopectin content in the mixture of polysaccharides is 30% or less, or 15% or less.

In some cases it may be desirable to use non-retrograding starches, such as waxy starch, in the current invention. The amount of amylopectin present in a starch may also be reduced by treating the starch with amylopectinase, which cleaves α-1,6 linkages resulting in the debranching of amylopectin into amylose.

In some cases a synthesis reaction can be carried out to prepare an amylose polymer having pendent coupling groups (for example, amylose with pendent ethylenically unsaturated groups) and steps may be performed before, during, and/or after the synthesis to enrich the amount of amylose, or purify the amylose.

Amylose of a particular size, or a combination of particular sizes can be used. The choice of amylose in a particular size range may depend on the application, for example, the type of polypeptide to be included, the desired size of the microparticle, or the like. In some embodiments amylose having an average molecular weight of 500,000 Da or less, 250,000 Da or less, 100,000 Da or less, 50,000 Da or less, preferably greater than 500 Da, or preferably in the range of about 1000 Da to about 100,000 Da is used. Amylose of particular molecular weights can be obtained commercially or can be prepared. For example, synthetic amyloses with average molecular masses of 70, 110, 320, and 1,000 kDa can be obtained from Nakano Vinegar Co., Ltd. (Aichi, Japan). The decision of using amylose of a particular size range may depend on factors such as the physical characteristics of the biodegradable composition (e.g., viscosity), the desired rate of degradation of the microparticle, the presence of other optional moieties in the biodegradable composition (for example, polypeptides, etc.), and the like.

Maltodextrin is typically generated by hydrolyzing a starch slurry with heat-stable α-amylase at temperatures of 85° C. to 90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate×100.

A starch preparation that has been totally hydrolyzed to dextrose (glucose) has a DE of 100, whereas starch has a DE of about zero. A DE of greater than 0 but less than 100 characterizes the mean-average molecular weight of a starch hydrolysate, and maltodextrins are considered to have a DE of less than 20. Maltodextrins of various molecular weights, for example, in the range of about 500 to about 5,000 Da are commercially available (for example, from CarboMer, San Diego, Calif.).

Another contemplated class of natural biodegradable polysaccharides is natural biodegradable non-reducing polysaccharides. A non-reducing polysaccharide can provide an inert matrix thereby improving the stability of sensitive polypeptides, such as proteins and enzymes. A non-reducing polysaccharide refers to a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). An exemplary non-reducing polysaccharide comprises polyalditol, which is available from GPC (Muscatine, Iowa). In another aspect, the polysaccharide is a glucopyranosyl polymer, such as a polymer that includes repeating (1→3)O-β-D-glucopyranosyl units.

In some aspects, the biodegradable compositions can include natural biodegradable polysaccharides that include chemical modifications other than the pendent coupling group. To exemplify this aspect, modified amylose having esterified hydroxyl groups can be prepared and used in biodegradable compositions in association with the methods of the invention. Other natural biodegradable polysaccharides having hydroxyl groups may be modified in the same manner. These types of modifications can change or improve the properties of the natural biodegradable polysaccharide making for a biodegradable composition that is particularly suitable for a desired application. Many chemically modified amylose polymers, such as chemically modified starch, have at least been considered acceptable food additives.

As used herein, “modified natural biodegradable polysaccharides” refers to chemical modifications to the natural biodegradable polysaccharide that are different than those provided by the coupling group or the initiator group. Modified amylose polymers having a coupling group (and/or initiator group) can be used in the compositions and methods of the invention.

To exemplify this aspect, modified amylose is described. By chemically modifying the hydroxyl groups of the amylose, the physical properties of the amylose can be altered. The hydroxyl groups of amylose allow for extensive hydrogen bonding between amylose polymers in solution and can result in viscous solutions that are observed upon heating and then cooling amylose-containing compositions such as starch in solution (retrograding). The hydroxyl groups of amylose can be modified to reduce or eliminate hydrogen bonding between molecules thereby changing the physical properties of amylose in solution.

Therefore, in some embodiments the natural biodegradable polysaccharides, such as amylose, can include one or more modifications to the hydroxyl groups wherein the modifications are different than those provided by a coupling group. Modifications include esterification with acetic anhydride (and adipic acid), succinic anhydride, 1-octenylsuccinic anhydride, phosphoryl chloride, sodium trimetaphosphate, sodium tripolyphosphate, and sodium monophosphate; etherification with propylene oxide, acid modification with hydrochloric acid and sulfuric acids; and bleaching or oxidation with hydrogen peroxide, peracetic acid, potassium permanganate, and sodium hypochlorite.

Examples of modified amylose polymers include carboxymethyl amylose, carboxyethyl amylose, ethyl amylose, methyl amylose, hydroxyethyl amylose, hydroxypropyl amylose, acetyl amylose, amino alkyl amylose, allyl amylose, and oxidized amylose. Other modified amylose polymers include succinate amylose and oxtenyl succinate amylose.

In another aspect of the invention, the natural biodegradable polysaccharide is modified with a hydrophobic moiety in order to provide a biodegradable matrix having hydrophobic properties. Exemplary hydrophobic moieties include those previously listed, fatty acids and derivatives thereof, and C₂-C₁₈ alkyl chains. A polysaccharide, such as amylose or maltodextrin, can be modified with a compound having a hydrophobic moiety, such as a fatty acid anhydride. The hydroxyl group of a polysaccharide can also cause the ring opening of lactones to provide pendent open-chain hydroxy esters. As an example, the natural biodegradable polysaccharide is a maltodextrin polymer comprising pendent acrylate or methacrylate groups, and pendent butyryl groups.

In some aspects, the hydrophobic moiety pendent from the natural biodegradable polysaccharide has properties of a therapeutic agent. The hydrophobic moiety can be hydrolyzed from the natural biodegradable polymer and released from the matrix to provide a therapeutic effect. One example of a therapeutically useful hydrophobic moiety is butyric acid, which has been shown to elicit tumor cell differentiation and apoptosis, and is thought to be useful for the treatment of cancer and other blood diseases. Other illustrative hydrophobic moieties include valproic acid and retinoic acid. Retinoic acid is known to possess antiproliferative effects and is thought to be useful for treatment of proliferative vitreoretinopathy (PVR). The hydrophobic moiety that provides a therapeutic effect can also be a natural compound (such as butyric acid, valproic acid, and retinoic acid). Therefore, degradation of the matrix having a coupled therapeutic agent can result in all natural degradation products.

In further aspects, the natural biodegradable polysaccharide can be modified with a corticosteroid. In these aspects, a corticosteroid, such as triamcinolone, can be coupled to the natural biodegradable polymer. One method of coupling triamcinolone to a natural biodegradable polymer is by employing a modification of the method described in Cayanis, E. et al., Generation of an Auto-anti-idiotypic Antibody that Binds to Glucocorticoid Receptor, The Journal of Biol. Chem., 261(11): 5094-5103 (1986). Triamcinolone hexanoic acid is prepared by reaction of triamcinolone with ketohexanoic acid; an acid chloride of the resulting triamcinolone hexanoic acid can be formed and then reacted with the natural biodegradable polymer, such as maltodextrin or polyalditol, resulting in pendent triamcinolone groups coupled via ester bonds to the natural biodegradable polymer.

Optionally, when the natural biodegradable polymer includes a pendent hydrophobic moiety and/or corticosteroid, the inventive compositions can further include an enzyme, such as lipase, to accelerate degradation of the bond between the hydrophobic moiety and the polysaccharide (e.g., ester bond).

According to the invention, a natural biodegradable polysaccharide that includes a coupling group can be used to form a microparticle core and/or a coating that is in contact with the core. Other polysaccharides can also be present in the biodegradable composition. For example, the two or more natural biodegradable polysaccharides can be used to form a microparticle. Examples include amylose and one or more other natural biodegradable polysaccharide(s), and maltodextrin and one or more other natural biodegradable polysaccharide(s); in one aspect the composition includes a mixture of amylose and maltodextrin, optionally with another natural biodegradable polysaccharide.

In one preferred embodiment, amylose or maltodextrin is the primary polysaccharide. In some embodiments, the composition includes a mixture of polysaccharides including amylose or maltodextrin and the amylose or maltodextrin content in the mixture of polysaccharides is 50% or greater, 60% or greater, 70% or greater, 80% or greater, or 85% or greater by weight.

Purified or enriched amylose preparations can be obtained commercially or can be prepared using standard biochemical techniques such as chromatography. In some aspects, high-amylose cornstarch can be used.

In some embodiments, the crosslinked polymeric coating on the microparticle core can be formed from a polymer other than a natural biodegradable polysaccharide. For example, a polymer formed from monomer or monomers including uncharged polar moieties can be used as polymeric material in the microparticle coating.

Suitable polymer backbones including uncharged polar moieties other than primary amide include polyethers (e.g., polyethylene glycol, polypropylene glycol), substituted polyalkylene imines (e.g., substituted polyethyleneimine), and the like. Compounds such as tetraethylene glycol, triethylene glycol, trimethylolpropane ethoxylate, and pentaerythritol ethoxylate can also be used.

Suitable pendant uncharged polar moieties include, for example substituted amide, ester, ether, sulfone, amine oxide, and the like. Suitable backbones for pendant uncharged polar moieties include alkyl, branched alkyl, polyether, and polyamine backbones, which can be formed from monomers such as vinyl monomers, acrylate ester monomers, secondary and tertiary acrylamide monomers, polyethylene glycol, polypropylene glycol, substituted polyethyleneimine, and the like.

The polymer, such as a biodegradable polysaccharide, is crosslinked to provide a polymeric matrix for controlling release of the polypeptide from the microparticles. Crosslinking can be accomplished by utilizing coupling groups that are associated with the polymer, such as coupling groups pendent from a natural biodegradable polysaccharide. As used herein, “coupling group” can include (1) a chemical group that is able to form a reactive species that can react with the same or similar chemical group to form a bond that is able to couple the polymers together (for example, wherein the formation of a reactive species can be promoted by an initiator); or (2) a pair of two different chemical groups that are able to specifically react to form a bond that is able to couple the polymers together. The coupling group can be attached to any suitable polymer, such as a natural biodegradable polysaccharide like amylose or maltodextrin polymers, which are exemplified herein. The polymers, once coupled, form polymer matrix.

Contemplated reactive pairs include Reactive Group A and corresponding Reactive Group B as shown in the Table 1 below. For the preparation of a composition, a reactive group from Group A can be selected and coupled to a first set of polymers and a corresponding reactive Group B can be selected and coupled to a second set of polymers. Reactive Groups A and B can represent first and second coupling groups, respectively. At least one and preferably two, or more than two reactive groups are coupled to an individual polymers. The first and second sets of polymers can be combined and reacted, for example, thermochemically, if necessary, to promote the coupling of polymers and the formation of a polymeric matrix.

TABLE 1

| Reactive group A | Reactive group B |
|---|---|
| amine, hydroxyl, sulfhydryl | N-oxysuccinimide (“NOS”) |
| amine | Aldehyde |
| amine | Isothiocyanate |
| amine, sulfhydryl | Bromoacetyl |
| amine, sulfhydryl | Chloroacetyl |
| amine, sulfhydryl | Iodoacetyl |
| amine, hydroxyl | Anhydride |
| aldehyde | Hydrazide |
| amine, hydroxyl, carboxylic acid | Isocyanate |
| amine, sulfhydryl | Maleimide |
| sulfhydryl | Vinylsulfone |

Amine also includes hydrazide (R—NH—NH₂).

For example, a suitable coupling pair would be an electrophilic group and a polymers having a nucleophilic group. An example of a suitable electrophilic-nucleophilic pair is N-hydroxysuccinimide-amine pair, respectively. Another suitable pair would be an oxirane-amine pair.

In some aspects, the polymers include at least one, and more typically more than one, coupling group per polymers, allowing for a plurality of polymers to be coupled in linear and/or branched manner. In some preferred embodiments, the polymers include two or more pendent coupling groups.

In some aspects, the coupling group on the polymer is a polymerizable group. In a free radical polymerization reaction the polymerizable group can couple polymers together in the composition, thereby forming a polymeric matrix.

A preferred polymerizable group is an ethylenically unsaturated group. Suitable ethylenically unsaturated groups include vinyl groups, acrylate groups, methacrylate groups, ethacrylate groups, 2-phenyl acrylate groups, acrylamide groups, methacrylamide groups, itaconate groups, and styrene groups. Combinations of different ethylenically unsaturated groups can be present on a polymer, such as a natural biodegradable polysaccharide like amylose or maltodextrin.

In preparing a polymer having pendent coupling groups any suitable synthesis procedure can be used. In the case of polymers containing hydroxyl groups, such as amylose or maltodextrin, suitable synthetic schemes typically involve reaction of the hydroxyl groups with a compound that can provide a pendent reactive coupling group. Synthetic procedures can be modified to produce a desired number of coupling groups pendent from the polymeric backbone. For example, the hydroxyl groups can be reacted with a coupling group-containing compound or can be modified to be reactive with a coupling group-containing compound. The number and/or density of coupling groups (such as acrylate groups) can be controlled using the present method, for example, by controlling the relative concentration of reactive moiety to monomer content.

In some modes of practice, the polymer, such as a biodegradable polysaccharide, has an amount of pendent coupling groups of about 0.7 μmoles of coupling group per milligram of polymer. In a preferred aspect, the amount of coupling group per polymer is in the range of about 0.3 μmoles/mg to about 0.7 μmoles/mg. For example, amylose or maltodextrin can be reacted with an acrylate groups-containing compound to provide an amylose or maltodextrin macromer having a acrylate group load level in the range of about 0.3 μmoles/mg to about 0.7 μmoles/mg.

In accordance with some aspects of the invention, the microparticle coating comprising the polymeric matrix, or microparticle with crosslinked natural biodegradable polysaccharide throughout, can be formed utilizing an initiator. As used herein, an “initiator” refers to a compound, or more than one compound, that is capable of promoting the formation of a reactive species from the coupling group of the polymer. For example, the initiator can promote a free radical reaction of polymers having coupling groups. In some embodiments, the initiator can be an “initiator polymer” that includes a polymer having a backbone and one or more initiator groups pendent from the backbone of the polymer.

Generally speaking, the initiator can be provided as a photoreactive group (photoinitiator) that is activated by radiation, or a redox initiator that is activated when members of a redox pair contact each other. Each of these aspects will now be described.

In some aspects the initiator is a compound that is light sensitive and that can be activated to promote the coupling of the polymers with pendent polymerizable groups via a free radical polymerization reaction. These types of initiators are referred to herein as “photoinitiators.” In some aspects it is preferred to use photoinitiators that are activated by light wavelengths that have no or a minimal effect on a polypeptide if present in the composition. A photoinitiator can be present in a polymeric composition independent of the polymer or pendent from a polymer.

In some embodiments, photoinitiation occurs using groups that promote an intra- or intermolecular hydrogen abstraction reaction. This initiation system can be used without additional energy transfer acceptor molecules and utilizing nonspecific hydrogen abstraction, but is more commonly used with an energy transfer acceptor, typically a tertiary amine, which results in the formation of both aminoalkyl radicals and ketyl radicals. Examples of molecules exhibiting hydrogen abstraction reactivity and useful in a polymeric initiating system, include analogs of benzophenone, thioxanthone, and camphorquinone.

In some preferred embodiments the photoinitiator includes one or more charged groups. The presence of charged groups can increase the solubility of the photoinitiator (which can contain photoreactive groups such as aryl ketones) in an aqueous system and therefore provide for an improved biodegradable composition. Suitable charged groups include, for example, salts of organic acids, such as sulfonate, phosphonate, carboxylate, and the like, and onium groups, such as quaternary ammonium, sulfonium, phosphonium, protonated amine, and the like. According to this embodiment, a suitable photoinitiator can include, for example, one or more aryl ketone photogroups selected from acetophenone, benzophenone, anthraquinone, anthrone, anthrone-like heterocycles, and derivatives thereof; and one or more charged groups, for example, as described herein. Examples of these types of water-soluble photoinitiators have been described in U.S. Pat. Nos. 5,714,360 and 6,077,698.

Other photoinitiators including one or more charged groups are described, for example, in U.S. Pat. Nos. 6,278,018 and 6,603,040.

Illustrative ionic or nonionic compounds having photoreactive moieties include tetrakis(4-benzoylphenylmethoxymethyl)methane (TBBE; as described in U.S. Pat. No. 5,414,075, see Example 1); 4,5-bis(4-benzoylphenylmethyleneoxy)benzene-1,3-disulfonic acid disodium salt (DBDS, Compound VI as described herein); and Ethylenebis(4-benzoylbenzyldimethylammonium)Dibromide (Diphoto-Diquat) (TEMED-DQ, Compound V as described herein) were used. Photogroup containing polymers include polysaccharides containing reactive groups (e.g., maltodextrin including sulphonate photoreactive groups); photopolyvinylpyrrolidone (also referred to as “photoPVP” and made as described in U.S. Pat. No. 5,002,582); PEI-APTAC-EITC initiator polymer (Compound I herein). Other photoreactive initiators such 4-benzoylbenzoic acid (BBA) groups, and 2,2′-azobis(2,4-dimethylvaleronitrile) can also be pendent from polymers.

In some aspects the photoinitiator is a compound that is activated by long-wavelength ultraviolet (LWUV) and visible light wavelengths. For example, in some aspects, the initiator includes a photoreducible or photo-oxidizable dye. Photoreducible dyes can also be used in conjunction with a compound such as a tertiary amine. The tertiary amine intercepts the induced triplet producing the radical anion of the dye and the radical cation of the tertiary amine. Examples of molecules exhibiting photosensitization reactivity and useful as an initiator include acridine orange, camphorquinone, ethyl eosin, eosin Y, erythrosine, fluorescein, methylene green, methylene blue, phloxime, riboflavin, rose bengal, thionine, and xanthine dyes. Use of these types of photoinitiators can be particularly advantageous when a light-sensitive polypeptide is included in the microparticle coating or microparticle forming composition.

In some aspects, the photoinitiator is a water soluble photoinitiator. A “water soluble” photoinitiator has a solubility in the composition of about 0.5% or greater.

In some embodiments, a water-soluble derivative of camphorquinone is utilized. Camphor or camphorquinone can be derivatized by techniques known in the art to add, for example, charged groups. See, for example, G. Ullrich et al. (2003) Synthesis and photoactivity of new camphorquinone derivatives;” Austrian Polymer Meeting 21, International H. F. Mark-Symposium, 131.

In some aspects of the invention, the water soluble photoinitiator is a diketone, which can be selected from water-soluble derivatives of camphoroquinone, 9,10-phenanthrenequinone, and naphthoquinone having an absorbance of 400 nm and greater. In some aspects of the invention, for example, the photoinitiator is a water-soluble non-aromatic alpha diketone, selected from water-soluble derivatives of camphorquinone.

Other suitable long-wave ultra violet (LWUV) or light-activatable molecules include, but are not limited to, [(9-oxo-2-thioxanthanyl)-oxy]acetic acid, 2-hydroxythioxanthone, and vinyloxymethylbenzoin methyl ether. Suitable visible light activatable molecules include, but are not limited to initiators comprising acridine orange, camphorquinone, ethyl eosin, eosin Y, Eosin B, erythrosine, fluorescein, methylene green, methylene blue, phloxime, riboflavin, rose bengal, thionine, xanthine dyes, and the like. In some embodiments, water soluble forms of visible light activatable molecules can be used.

As mentioned above, the initiator can comprise a photoinitiator or a redox initiator. Thus, in some aspects, the initiator includes an oxidizing agent/reducing agent pair, a “redox pair,” to drive polymerization of the polymeric material. In this case, polymerization of the polymers is carried out upon combining one or more oxidizing agents with one or more reducing agents. In general, combinations of organic and inorganic oxidizers, and organic and inorganic reducing agents are used to generate radicals for polymerization. A description of redox initiation can be found in Principles of Polymerization, 2^{nd} Edition, Odian G., John Wiley and Sons, pgs 201-204, (1981). Other compounds can be included in the composition to promote polymerization of the polymers.

When combined, the oxidizing agent and reducing agent can provide a particularly robust initiation system and can drive the formation of a polymerized matrix of polymers from a composition having a low viscosity. A polymer composition with a low viscosity may be due to a low concentration of polysaccharide in the composition, a polysaccharide having a low average molecular weight, or combinations thereof.

In order to promote polymerization of the polymers in a composition to form a matrix, the oxidizing agent is added to the reducing agent in the presence of the one or more polymers. For example, a composition including a polymer and a reducing agent is added to a composition including an oxidizing agent, or a composition including a polymer and an oxidizing agent is added to a composition containing a reducing agent. One desirable method of preparing a matrix is to combine a composition including a polymer and an oxidizing agent with a composition including a polymer and a reducing agent. For purposes of describing this method, the terms “first composition” and “second composition” can be used.

The viscosities of first and second compositions can be the same or can be different. Generally, though, it has been observed that good mixing and subsequent matrix formation is obtained when the compositions have the same or similar viscosities. In this regard, if the same polymer is used in the first and second compositions, the concentration of the polymer may be the same or different.

The oxidizing agent can be selected from inorganic or organic oxidizing agents, including enzymes; the reducing agent can be selected from inorganic or organic reducing agents, including enzymes. Exemplary oxidizing agents include peroxides, including hydrogen peroxide, metal oxides, and oxidases, including glucose oxidase. Exemplary reducing agents include salts and derivatives of electropositive elemental metals such as Li, Na, Mg, Fe, Zn, Al, and reductases. In one mode of practice, the reducing agent is present at a concentration of about 2.5 mM or greater when the reducing agent is mixed with the oxidizing agent. Prior to mixing, the reducing agent can be present in a composition at a concentration of, for example, 5 mM or greater.

Other polymerization promoting compounds can be included in the composition, such as metal or ammonium salts of persulfate.

In some aspects the polymerization initiator (photoinitiator or redox initiator) is a polymer that includes an initiator group (herein referred to as an “initiator polymer”). The polymeric portion of the initiator polymer can be obtained or prepared to have particular properties or features that are desirable for use with a microparticle coating or microparticle forming composition. For example, the polymeric portion of the initiator polymer can have hydrophilic or amphoteric properties, or it can include pendent charged groups. Optionally, or additionally, the polymer can change or improve the properties of the matrix that is formed by the polymer having coupling groups. For example, the initiator polymer can change the elasticity, flexibility, wettability, or softness (or combinations thereof) of the polymeric matrix. Certain polymers, as described herein, are useful as plasticizing agents for matrix-forming compositions. Initiator groups can be added to these plasticizing polymers and used in the compositions and methods of the invention.

For example, in some aspects an initiator can be pendent from a polymer. Therefore, the polymer with the initiator group is able to promote activation of polymerizable groups that are pendent from other polymers and promote the formation of a crosslinked matrix.

In other cases, the polymeric portion of the initiator polymer can include, for example, acrylamide and methacrylamide monomeric units, or derivatives thereof. In some embodiments, the coating composition includes an initiator polymer having a photoreactive group and a polymeric portion selected from the group of acrylamide and methacrylamide polymers and copolymers.

In still further embodiments, the initiator can be present as an independent component of the composition used to form the crosslinked matrix. The initiator can be present in the composition at a concentration sufficient for matrix formation. In some aspects, the initiator (for example, a water soluble non-aromatic alpha diketone such as a water soluble camphorquinone derivative) is used at a concentration of about 0.5 mg/mL or greater. In some aspects, the water soluble photoinitiator can be present at a concentration in the range of about 0.1 mg/mL to about 10 mg/mL.

Other suitable charged polymerization initiators are described, for example, in U.S. Publication No. 2004/0202774, Chudzik et al., “Charged initiator polymers and methods of use.”

In accordance with these aspects, the initiator polymer can include light-activated photoinitiator groups, thermally activated initiator groups, chemically activated initiator groups, or combinations thereof. Suitable thermally activated initiator groups include 4,4′ azobis(4-cyanopentanoic) acid and 2,2-azobis[2-(2-imidazolin-2-yl)propane]dihydrochloride or other thermally activated initiators provided these initiators can be incorporated into an initiator polymer. Chemically activated initiation is often referred to as redox initiation, redox catalysis, or redox activation. In general, combinations of organic and inorganic oxidizers, and organic and inorganic reducing agents are used to generate radicals for polymerization. Illustrative redox initiators are described herein. In some embodiments, it is useful to utilize redox initiators that are not damaging to biological systems. In some embodiments, it is useful to utilize photoinitiator groups and thermally activated initiator groups that utilize energy that is not damaging to biological systems. In one embodiment, photoinitiator groups having long wavelength UV and visible light-activated frequencies are coupled to the backbone of the initiator polymer. In one embodiment, visible light-activated photoinitiators are coupled to the polymer backbone. Any of the thermally reactive, photoreactive, and/or redox initiators described herein can be used.

In one embodiment, photoinitiator groups having an absorbance of 350 nm and greater are used. In some aspects, photoinitiator groups having an absorbance of 500 nm and greater are used. Suitable photoinitiator groups include light-activated initiator groups, such as long-wave ultra violet (LWUV) light-activatable molecules and visible light activatable molecules, as described elsewhere herein.

The positive charge of the cationic portion of the initiator polymer can be contributed by the backbone of the initiator polymer, by positively-charged groups pendent from the backbone, or both. In one embodiment, the initiator polymer has a plurality of cationic groups pendent from the backbone of the initiator polymer; in some aspects, the cationic groups can be provided by ternary or quaternary cationic moieties, such as quaternary amine groups. In another embodiment the polymeric backbone contains nitrogen and can be, for example, a polymeric imine.

In some embodiments, the initiator polymer has a polymeric backbone that is coupled to at least one and more typically a plurality of cationic groups. The polymer backbone, which generally refers to the polymer chain without addition of any initiator group or cationic group, typically includes carbon and preferably one or more atoms selected from nitrogen, oxygen, and sulfur. The backbone can include carbon-carbon linkages and, in some embodiments, can also include one or more of amide, amine, ester, ether, ketone, peptide, or sulfide linkages, or combinations thereof. Examples of suitable polymer backbones include polyesters, polycarbonates, polyamides, polyethers (such as polyoxyethylene), polysulfones, polyurethanes, or copolymers containing any combination of the representative monomer groups.

The polymeric backbone can include reactive groups useful for the coupling of cationic groups to form the initiator polymer. Suitable reactive groups include acid (or acyl) halide groups, alcohol groups, aldehyde groups, alkyl and aryl halide groups, amine groups, carboxyl groups, and the like. These pendent reactive groups can be used for coupling the initiator group and, in some embodiments, for coupling of the cationic groups to the polymeric backbone. These chemical groups can be present either on a preformed polymer or on monomers used to create the positively-charged initiator polymer. Examples of polymers having suitable reactive or charged side group include polymers, and in particular dendrimers, having reactive amine groups such as polylysine, polyornithine, polyethylenimine, and polyamidoamine.

In one embodiment of the invention, the backbone of the initiator polymer provides an overall positive charge and contributes to the cationic portion. An example of this type of polymeric backbone includes polymers having imine linkages, such as polyimines that also include primary, secondary, or tertiary amine groups. Use of these types of polymers in the synthesis of the initiator polymer are preferred as they can provide a highly derivatizable preformed polymer backbone to which a plurality of cationic groups and initiator groups can be coupled. Polyamines that are particularly suitable as a starting polymer for the synthesis of the initiator polymer include polyethylenimine, polypropylenimine, and the like, and polyamine polymers or copolymers, and in particular dendrimers, formed from monomers such as the following amine functional monomers: 2-aminomethylmethacrylate, 3-(aminopropyl)-methacrylamide, and diallylamine. Suitable polyamines are commercially available, for example, Lupasol™ PS (polyethylenimine; BASF, New Jersey).

In some embodiments, the backbone of the initiator polymer is coupled to one or more cationic groups. Illustrative cationic groups have a stable positive charge and include ternary and quaternary cationic groups. In some embodiments, cationic groups include quaternary ammonium, quaternary phosphonium, and ternary sulfonium. These groups can be provided in, for example, alkylated or alkoxylated forms having, for example, in the range of 1-6 carbons on each chain. Examples include, but are not limited to tetraalkylammonium, tetraalkoxyammonium, trialkylsulfonium, trialkoxysulfonium, tetraalkylphosphonium, and tetraalkoxyphosphonium cations. Specific examples include tetramethylammonium, tetrapropylammonium, tetrabenzylammonium and the like.

Optionally, the compositions and methods of the invention can include polymerization accelerants that can improve the efficiency of polymerization. Examples of useful accelerants include N-vinyl compounds, particularly N-vinyl pyrrolidone and N-vinyl caprolactam. Such accelerants can be used, for instance, at a concentration of between about 0.01% and about 5%, and preferably between about 0.05% and about 0.5%, by weight, based on the volume of the microparticle coating or microparticle forming composition.

In some aspect of the invention, a natural biodegradable polysaccharide that includes a coupling group is used to form a microparticle core or a coating in contact with the core. Other polysaccharides can also be present in the biodegradable composition. For example, the composition can include two different natural biodegradable polysaccharides, or more than two different natural biodegradable polysaccharides. For example, in some cases the natural biodegradable polysaccharide (such as amylose or maltodextrin) can be present in the composition along with another biodegradable polymer (i.e., a secondary polymer), or more than one other biodegradable polymer. An additional polymer or polymers can be used to alter the properties of the matrix, or serve as bulk polymers to alter the volume of the matrix formed from the biodegradable composition. For example, other biodegradable polysaccharides can be used in combination with the amylose polymer. These include hyaluronic acid, dextran, starch, amylose (for example, non-derivatized), amylopectin, cellulose, xanthan, pullulan, chitosan, pectin, inulin, alginates, and heparin.

In some aspects of the invention, a composition that includes at least the natural biodegradable polysaccharide (such as amylose or maltodextrin having a coupling group), and a polypeptide, is used to form a microparticle. In some embodiments the composition includes the natural biodegradable polysaccharide, a polypeptide, and an initiator.

The concentration of the natural biodegradable polysaccharide in the composition can be chosen to provide a microparticle having a desired density of crosslinked natural biodegradable polysaccharide. In some embodiments, the concentration of natural biodegradable polysaccharide in the composition can depend on the type or nature of the polypeptide that is included in the composition. In some embodiments the natural biodegradable polysaccharide having the coupling groups is present in the microparticle at a concentration in the range of about 5% to about 95% (w/v), or about 5% to about 90%, or in the range of about 5% to about 85% and in other embodiments in the range of about 10% to about 80% (w/v). In some embodiments, the amount of the polypeptide solution provided to the microparticles has a polypeptide concentration in the range of about 0.5 to about 4 mg.

In some aspects, the concentration of polysaccharide in the microparticle can be characterized relative to the concentration of polypeptide in the microparticle. For example, the polysaccharide can comprise maltodextrin, and the microparticle can have a polypeptide-to-maltodextrin ratio of about 2:1.

Other polymers or non-polymeric compounds can be included in the composition that can change or improve the properties of the microparticle that is formed by the natural biodegradable composition having coupling groups in order to change the elasticity, flexibility, wettability, or adherent properties, (or combinations thereof) of the microparticle.

The microparticle with a core composed predominantly of polypeptide and a coating including a crosslinked polymeric coating can be formed in various ways according to the invention. A first general method of forming these microparticles involves initially providing a “core” polypeptide microparticle and then forming a crosslinked polymeric coating on the core. A second general method involves initially providing a composition that includes polypeptide, nucleation agent, and polymeric material used to form the coating, and then performing particular steps which results in a polypeptide microparticle having a polypeptide core-crosslinked polymeric coating structure.

For the first general method, any type of “core” polypeptide microparticle that is formed predominantly of polypeptide can be used. For example, freeze or spray-drying techniques have been carried out which provide polypeptide microparticles, which are suitable for use as the core particles in the methods of the invention. In preferred modes of practice, the polypeptide microparticles can be formed as described in “Polypeptide Microparticles,” Slager et al., U.S. Ser. No. 60/937,492, filed Jun. 28, 2007.

In some aspects, the invention provides a method for forming a microparticle comprising a core comprising predominantly polypeptide and a microparticle coating comprising a crosslinked polymeric matrix. The method includes the steps of: (a) in a liquid composition, providing a core particle comprising predominantly polypeptide; (b) mixing the core particle with a first component comprising a first reactive group; (c) mixing the core particle with a second component comprising a polymer and a pendent a second reactive group; wherein either: (i) the first reactive group is reactive with the second reactive group, thereby forming the crosslinked polymeric matrix, or (ii) the first reactive group comprises a polymerization initiator group and the second reactive group comprises a polymerizable group, and the method additionally comprises (d) activating the initiator group to cause polymerization of the first component, thereby forming the crosslinked polymeric matrix, and wherein step (b) can be performed before, after, or at the same time as step (c).

In many modes of practice the first reactive group includes a polymerization initiator, and the second reactive group (which is pendent from the polymer) comprises a polymerizable group, such as an ethylenically unsaturated group.

In many aspects the method also includes a step of adding a phase separation agent to the liquid composition. In many modes of practice, the concentration of the phase separation agent in the range of 100 mg/mL to 500 mg/mL.

In some embodiments, the phase separation agent can be combined with the polymerization initiator prior to addition of the initiator to the polypeptide microparticles. In some embodiments, the phase separation agent can be combined with polymer with pendent reactive groups to form a coating solution, and the coating solution is then combined with the composition containing the core particles. In these aspects, the phase separation agent can serve as a solvent for the polymerization initiator or the polymer, respectively. When utilized in this manner, the phase separation agent can assist in localizing (e.g., coalescing) components of the system (e.g., by water exclusion), thereby enhancing efficacy of the inventive methods.

In preparation for coating the core particles, the core particles are suspended in a suitable solvent, such as an organic solvent. Illustrative organic solvents include chloroform, dichloromethane, acetone, isopropyl alcohol, or the like. The solvent can be selected based upon such factors as the composition of the microparticles, the composition of the coating composition to be applied to the core particles, and the like.

The core particles suspensions are contacted with a polymerization initiator. In some embodiments, the initiator is present in solution with an organic solvent, such as methanol, chloroform, dichloromethane, acetone, isopropyl alcohol, combinations of any two or more of these, and the like. When the initiator is a charged initiator that is also water soluble, the initiator can be provided as an aqueous solution of initiator.

The concentration of initiator in solvent (organic or aqueous) can vary depending upon the particular initiator and solvent selected. Illustrative concentrations for the initiator in solvent (organic or aqueous) include about 0.1 mg/ml to about 20 mg/ml or about 0.5 mg/ml to about 1 mg/ml.

In some aspects, the polymerization initiator can be provided in solution with the phase separation agent. Combination of the initiator and phase separation agent can provide advantages. It is believed the phase separation agent can assist in concentrating or coalescing the initiator at the surface of the polypeptide microparticle “core.” When crosslinkable polymer (biodegradable polysaccharide or other polymer) is subsequently provided to the microparticles having initiator localized at their surface, crosslinking of the polymer can be more efficiently performed.

The concentration of initiator in the phase separation agent can vary depending upon the particular initiator selection and phase separation agent utilized. In some embodiments, the initiator is a charged initiator, and the phase separation agent is PEG. In these embodiments, illustrative concentration of initiator in phase separation agent can be in the range of about 0.1 mg/ml to about 10 mg/ml, or about 0.5 mg/ml to about 1 mg/ml. In some embodiments, the initiator can comprise redox initiator, and the phase separation agent is PEG. In these embodiments, illustrative concentration of initiator (i.e., sodium persulphate) in phase separation agent can be in the range of about 1 mg/ml to about 100 mg/ml, or about 40 mg/ml to about 60 mg/ml.

Any of the initiators described herein can be utilized in connection with these aspects of the invention.

In some modes of practice, subsequent to contacting the microparticles with polymerization initiator, a coating solution is applied to the microparticles/polymerization initiator. The coating solution can comprise polymer containing pendent polymerizable groups. The polymer can be a degradable polysaccharide containing polymerizable groups as described herein

In some modes of practice, crosslinkable polymer is provided to the microparticles in a concentration sufficient to provide a coating at the surface of the microparticle core. Illustrative concentrations of polymer, such as biodegradable polysaccharide, are in the range of about 5 mg/mL to about 1000 mg/mL or about 50 mg/mL to about 300 mg/mL.

In one desired mode of practice, the crosslinkable polymer is added to the microparticles with good mixing to thoroughly combine the components. The mode of mixing (e.g., agitation) can be chosen based upon factors such as the concentration of microparticles and biodegradable polysaccharide, and the like. Such agitation can be performed using vortexing equipment, through use of stirring equipment such as stir bars, or by manually shaking the receptacle. Mixing is generally carried out until the microparticles and the biodegradable polysaccharide are sufficiently combined, which may only take a few seconds, or may be longer for larger volumes.

The initiator can then be activated to couple the polymer and thereby form a coating comprising a crosslinked polymeric matrix on the microparticle core. The coating serves as a polypeptide release controlling coating. Activation conditions will depend upon the particular initiator selected. In some embodiments, the initiator selected is a charged initiator containing photoreactive compounds. Illustrative activation conditions are included in the Examples herein. In some embodiments, the initiator selected is a redox initiator, and illustrative conditions for activation of the redox initiator are included in the Examples herein.

In other modes of practice the first reactive group is reactive with the second reactive group, thereby forming the crosslinked polymeric matrix. For example, the coating can be formed using a component such as a first polymer with a first reactive group, and second polymer comprising a pendent second reactive group, wherein the first and second groups are reactive (e.g., thermochemically reactive) to form the crosslinked matrix.

The coating can be formed by using a contacting the core particle with a component comprising a first reactive group. As an example, the first component can be a polymer containing a first reactive group such as an amine group. One example is a PEI polymer having pendent quaternary amine groups and pendent hydrophobic groups. After the first component is coated on the core, a next step can be performed wherein the microparticle is contacted with a second polymer with a second reactive group. The second reactive group reacts with the first reactive group and results in a crosslinked matrix that includes, for example, the first and second polymers.

In some embodiments, the first or second polymer can comprise monomer or monomers including uncharged polar moieties. Suitable polymer backbones including uncharged polar moieties include polyethers (e.g., polyethylene glycol, polypropylene glycol), substituted polyalkylene imines (e.g., substituted polyethyleneimine), and the like. Suitable pendant uncharged polar moieties include, for example substituted amide, ester, ether, sulfone, amine oxide, and the like. Suitable backbones for pendant uncharged polar moieties include alkyl, branched alkyl, polyether, and polyamine backbones, which can be formed from monomers such as vinyl monomers, acrylate ester monomers, secondary and tertiary acrylamide monomers, polyethylene glycol, polypropylene glycol, substituted polyethyleneimine, and the like. One illustrative polymer is N,N-disubstituted acrylamide. Illustrative polymers in accordance with these aspects of the invention are described in US 2005/0074478 Al, “Attachment of Molecules to Surfaces,” Ofstead et al.

In some aspects, the first or second polymer can comprise a hydrophilic polymer, such as polyethylene glycol. The first or second polymer can be crosslinked via coupling groups to form a polymeric matrix. For example, the matrix can be formed from the crosslinking of aminated-polyalditol with CDI-modified PEG. Aminated-polyalditol, CDI-modified PEGs of various molecular weights (3350 Da, 2000 Da, 1500 Da, 1000 Da, 600 Da (ave. mol. wt.)), CDI-modified tetraethylene glycol, CDI-modified triethylene glycol, CDI-modified trimethylolpropane ethoxylate (20 EO), and CDI-modified pentaerythritol ethoxylate (15 EO)_are described in U.S. Pub. No. 2008/0039931 (U.S. application Ser. No. 11/789,786), “Hydrophilic Shape Memory Insertable Medical Articles,” filed Apr. 25, 2007.

In another aspect of the invention, the polypeptide microparticles include a (i) a core comprising predominantly polypeptide; and (ii) a microparticle coating comprising a polymer comprising pendent hydrophobic groups. The polymer adheres to the core and is able to modulate release of the polypeptide from the microparticle. In this embodiment, the polymeric material of the coating is not required to be crosslinked.

The polymer can have a backbone formed of monomers including uncharged polar moieties, such as those described herein. In some aspects the polymer comprising pendent hydrophobic groups also comprises a poly(ethyleneimine) backbone. In some aspects the polymer comprising pendent hydrophobic groups having a molecular weight of 250,000 Da or less.

The hydrophobic groups that are pendent from the backbone of the polymer can allow the polymer to adhere to the microparticle core. Exemplary hydrophobic groups can be derived from organic dyes, such as eosin. Exemplary hydrophobic groups can also include structures having heterocyclic rings fused with benzenoid rings.

The microparticles can be formed by a method comprising the steps of providing a core particle comprising predominantly polypeptide in a liquid composition, and mixing the core particle with a polymer comprising pendent hydrophobic groups

In some preferred aspects, the method includes one or more of the following additional step(s) or feature(s): mixing the polymer comprising pendent hydrophobic groups is with the core particle at a weight ratio in the range of 100:0.5 to 100:5. Since substantially all of the polymer can be adhered to the microparticle core, the weight ratio of the core to the microparticle coating in the coated microparticle can be in the range of 100:0.5 to 100:5. In some aspects the method is carried out using a composition comprising a halogenated solvent.

In some aspects, polypeptide microparticles are formed by combining polypeptide with a natural biodegradable polysaccharide. The biodegradable polysaccharide is then crosslinked, thereby forming a matrix that incorporates the polypeptide. This results in a microparticle that comprises a crosslinked matrix of biodegradable polysaccharide, and a polypeptide incorporated in the crosslinked matrix. For forming the microparticle, a biodegradable polysaccharide that has a molecular weight of 500,000 Da or less is used. Also, microparticle comprises a ratio of polypeptide to biodegradable polysaccharide in the range of 3:1 to 1:3 by weight. The crosslinked matrix also comprises reacted polymerizable groups that covalently couple biodegradable polysaccharide together. Preferably, the reacted polymerizable groups comprise reacted methacrylate groups, that the reacted polymerizable groups are pendent from the biodegradable polysaccharide in an amount in the range of DS 0.1 to DS 0.5; and/or that the biodegradable polysaccharide has a molecular weight in the range of 1,000 Da to 100,000 Da.

The invention also provides methods of preparing microparticles that comprise: (a) a crosslinked matrix of biodegradable polysaccharide, and (b) polypeptide incorporated in the crosslinked matrix. Generally speaking, once the biodegradable polysaccharide and polypeptide(s) have been combined, the polysaccharide is polymerized to form a matrix that incorporates (e.g., entraps) the polypeptide. An initiator is utilized that is capable of promoting the formation of a reactive species from the coupling group. The initiator can be provided as a photoinitiator or a redox initiator. Polymerization initiation will thus depend upon the particular initiator(s) chosen. Polymerization of the polysaccharide can be induced by a variety of means such as irradiation with light of suitable wavelength, or by contacting members of a redox pair.

Thus, in some embodiments, the invention relates to methods for preparing a microparticle comprising steps of:
(a) in solution, combining polypeptide and biodegradable polysaccharide to provide a polypeptide composition;
(b) combining a phase separation agent with the polypeptide composition;
(c) combining a polymerization initiator with the polypeptide composition; and
(d) activating the initiator to couple the biodegradable polysaccharides, thereby forming microparticles comprising a crosslinked matrix of biodegradable polysaccharide and polypeptide incorporated in the crosslinked matrix.

In accordance with these aspects, a solution comprising polypeptide and biodegradable polysaccharide is prepared. Generally, the polypeptide is provided as an aqueous solution. The preparation of this aqueous solution may involve, for example, the solubilization of a lyophilized polypeptide, or the dilution of a concentrated solution of polypeptide with an aqueous solution. The polypeptide solution can be prepared as an aqueous buffered solution. Exemplary buffers include sodium phosphate (e.g., phosphate-buffered saline), and 2(N-morpholino) ethanesulfonic acid (MES), which can be used at concentrations of about 5 mM in the polypeptide solution.

The polypeptide is dissolved in solution at a concentration sufficient for the formation of polypeptide microparticles with biodegradable polysaccharide. In many preparations, the concentration of polypeptide in solution is generally about 20 mg/ml or greater. However, lower concentrations of polypeptide can be used in some embodiments. In some specific modes of practice, the polypeptide is an antibody or Fab fragment, which is in solution at a concentration in the range of about 10 mg/ml to about 50 mg/ml, and more specifically in the range of about 20 mg/ml to about 25 mg/ml.

Once prepared, the polypeptide solution is then combined with one or more selected biodegradable polysaccharides. The biodegradable polysaccharide is typically provided at a concentration sufficient to provide a microparticle having structural integrity. Illustrative concentrations of the biodegradable polysaccharide are in the range of about 0.5 mg/ml to about 50 mg/ml, or about 0.5 mg/ml to about 25 mg/ml, or about 0.5 mg/ml to about 1 mg/ml.

Once combined, the resulting polypeptide/polysaccharide solution can have a polysaccharide concentration as described elsewhere herein. The relative amounts of polypeptide and polysaccharide can be selected to provide a desired polypeptide:polysaccharide ratio as described elsewhere herein.

In accordance with the inventive method, a phase separation agent is combined with the polypeptide composition. The phase separation agent is a compound capable of being dissolved in both aqueous and organic solvents, and that can promote formation of the polypeptide microparticles. More particularly, the phase separation agent is a compound capable of being dissolved in a solvent such as chloroform or dichloromethane, as well as in an aqueous solvent, and which can be separated from the polypeptide microparticles after they are formed, if desired. In some embodiments, the phase separation agent can be an amphiphilic compound.

In some aspects, a concentrated solution of a phase separation agent (such as an amphiphilic polymer) is prepared and then added to the polypeptide composition. In many modes of practice, the phase separation agent is added to the polypeptide composition in an initial concentration of about 30% (w/v) to achieve a final concentration of the phase separation agent of about 7% (w/v) or greater. In some aspects the final concentration of the phase separation agent can be in the range of about 2% (w/v) to about 20% (w/v), or about 5% (w/v) to about 10% (w/v). For example, a phase separation agent such as PEG can be used in the initial concentration of about 30% (w/v) and a final concentration of about 7.7% (w/v).

When the phase separation agent comprises an amphiphilic compound, the amphiphilic compound can be selected from polymeric and non-polymeric amphiphilic materials. In some aspects, the amphiphilic compound is an amphiphilic polymer.

Exemplary amphiphilic polymers and compounds include poly(ethyleneglycol) (PEG) and PEG copolymers, tetraethylene glycol, triethylene glycol, trimethylolpropane ethoxylate, and pentaeerythritol ethoxlylate, polyvinylpyrrolidone (PVP) and PVP copolymers, dextran, Pluronic, polyacrylic acid, polyacrylamide, polyvinyl pyridine, polylysine, polyarginine, PEG sulfonates, fatty quaternary amines, fatty sulfonates, fatty acids, dextran, dextrin, and cyclodextrin. The amphiphilic polymer can also be a copolymer containing hydrophilic and hydrophobic polymeric blocks.

In one desired mode of practice, the polypeptide composition and the phase separation agent are combined with good mixing to thoroughly combine the components. The mode of mixing (e.g., agitation) can be chosen based on the factors such as the size of the receptacle containing the phase separation agent and the polypeptide composition. Such agitation can be performed using vortexing equipment, through use of stirring equipment such as stir bars, or by manually shaking the receptacle. Mixing is generally carried out until the phase separation agent and the biodegradable polysaccharide/polypeptide are sufficiently combined, which may only take a few seconds, or may be longer for larger volumes.

During and after mixing, polypeptide is coalesced with the biodegradable polysaccharide for microparticle formation. The polypeptide is further coalesced by the principle of water exclusion. The phase separation agent sequesters the water molecules and drives the polypeptide to coalesce with the biodegradable polysaccharide.

In accordance with the method, polymerization initiator is combined with the polypeptide composition. The polymerization initiator can be added alone, or in combination with the phase separation agent.

Suitable polymerization initiators are discussed elsewhere herein.

In some embodiments, the polymerization initiator and phase separation agent can be combined to form an initiator solution. The initiator solution can then be combined with the polypeptide composition. Combination of the initiator and phase separation agent prior to adding these components to the polypeptide composition can provide advantages for crosslinking the biodegradable polysaccharide. The phase separation agent can assist in concentrating or coalescing the initiator with the biodegradable polysaccharide and polypeptide, thereby bringing these components in proximity to each other prior to crosslinking of the biodegradable polysaccharide. In a subsequent step, the initiator can be activated to couple the biodegradable polysaccharide, thereby forming microparticles comprising a crosslinked matrix of biodegradable polysaccharide and polypeptide incorporated in the crosslinked matrix.

The concentration of initiator in the phase separation agent can vary depending upon the particular initiator selection and phase separation agent utilized. In some embodiments, the initiator is a charged initiator, and the phase separation agent is PEG. In these embodiments, illustrative concentration of initiator in phase separation agent can be in the range of about 0.1 mg/ml to about 10 mg/ml, or about 0.5 mg/ml to about 1 mg/ml. In some embodiments, the initiator can comprise redox initiator, and the phase separation agent can comprise PEG. In these embodiments, illustrative concentration of a redox initiator (such as sodium persulphate) in phase separation agent can be in the range of about 10 mg/ml to about 100 mg/ml, or about 40 mg/ml to about 50 mg/ml. In some embodiments, it can be desirable to include a redox initiator that has the potential to react with the polypeptide in the phase separation agent. In these aspects, interaction with the polypeptide (such as by oxidizing the polypeptide) can be minimized or avoided.

In one desired mode of practice, the polymerization initiator (whether combined alone, or as an initiator solution containing phase separation agent) is added to the polypeptide composition with good mixing to thoroughly combine the components. The mode of mixing (e.g., agitation) can be chosen based on the factors such as the size of the receptacle containing the polymerization initiator and the polypeptide composition. Such agitation can be performed using vortexing equipment, through use of stirring equipment such as stir bars, or by manually shaking the receptacle. Mixing is generally carried out until the polymerization initiator and the biodegradable polysaccharide/polypeptide are sufficiently combined, which may only take a few seconds, or may be longer for larger volumes.

The concentration of polymerization initiator is sufficient to provide adequate crosslinking of the biodegradable polysaccharide. Final concentration of polymerization initiator in the polypeptide composition can be in the range of about 0.1 mg/ml to about 10 mg/ml.

Once the above components have been combined (polypeptide, biodegradable polysaccharide, phase separation agent, polymerization initiator), the initiator is activated to couple the biodegradable polysaccharides, thereby forming microparticles comprising a crosslinked matrix of biodegradable polysaccharide and polypeptide incorporated in the crosslinked matrix.

In some aspects the initiator is a compound that is light sensitive and that can be activated to promote the coupling of the polysaccharide via a free radical polymerization reaction (“photoinitiators”). In some aspects it is preferred to use photoinitiators that are activated by light wavelengths that have no or a minimal effect on polypeptide of interest.

In some modes of practice, in order to promote polymerization of the biodegradable polysaccharides in a composition to form a matrix, an oxidizing agent is added to a reducing agent in the presence of the one or more biodegradable polysaccharides. These methodologies thus involve the use of a redox pair to initiate polymerization of the polysaccharides, thereby forming a polysaccharide matrix. The polysaccharide matrix forms a microparticle that is capable of incorporating and delivering polypeptide as described herein. For example, a reducing agent and oxidizing agent can be separately (sequentially) added to a polypeptide composition.

The initiator can then be activated to couple the biodegradable polysaccharide and thereby form a crosslinked matrix of biodegradable polysaccharide and polypeptide incorporated in the crosslinked matrix. Activation conditions will depend upon the particular initiator selected. In some embodiments, the initiator selected is a charged initiator containing photoreactive compounds. Illustrative activation conditions are included in the Examples herein. In some embodiments, the initiator selected is a redox initiator, and illustrative conditions for activation of the redox initiator are included in the Examples herein.

Optionally, the formed polypeptide microparticles can be subjected to a step of cooling following the polymerization of biodegradable polysaccharide. In the cooling step, the agitated mixture is brought down to a temperature, eventually, that solidifies the mixture by freezing (such as below 0° C.). The microparticle preparation is kept at this low temperature until completely frozen. During the cooling process, and prior to freezing, there may be further aggregation of the free polypeptide with the biodegradable polysaccharide.

The microparticles can be kept frozen before the microparticles are further processed to remove the phase separation agent. Prior to removal of the phase separation agent, the microparticle preparation can be treated to remove the water content in the preparation. The treatment can be a drying step, which can be carried out by a process such as vacuum drying or lyophilization.

The lyophilized microparticles can then optionally be subjected to removal of the phase separation agent. In one mode of practice, the dried microparticle preparation is treated (for example, by washing) with an organic solvent, such as chloroform, dichloromethane, acetone, isopropyl alcohol, or the like, to remove the phase separation agent. Repeated washes of the dried lyophilized microparticle preparation can be performed to remove predominantly all of the phase separation agent from the microparticles. The washing steps can be carried out at a desired temperature (e.g., room temperature).

Following washes, the microparticles can be stored in dried form, and for example, frozen until prepared for use.

The formed microparticles thus include a crosslinked matrix of biodegradable polysaccharide and a polypeptide incorporated in the crosslinked matrix. In accordance with these aspects, the release of polypeptide from the microparticles is controlled by the crosslinked matrix that forms the microparticle.

Optionally, the microparticle of the invention can also include one or more additional components such as biodegradable polymers. Examples of biodegradable polymers that can be included in the microparticle include, for example, polylactic acid, poly(lactide-co-glycolide), polycaprolactone, polyphosphazine, polymethyldienemalonate, polyorthoesters, polyhydroxybutyrate, polyalkeneanhydrides, polypeptides, polyanhydrides, and polyesters, and the like.

Other additional biodegradable polymers include biodegradable polyetherester copolymers. Generally speaking, the polyetherester copolymers are amphiphilic block copolymers that include hydrophilic (for example, a polyalkylene glycol, such as polyethylene glycol) and hydrophobic blocks (for example, polyethylene terephthalate). Examples of block copolymers include poly(ethylene glycol)-based and poly(butylene terephthalate)-based blocks (PEG/PBT polymer). Examples of these types of multiblock copolymers are described in, for example, U.S. Pat. No. 5,980,948. PEG/PBT polymers are commercially available from Octoplus BV, under the trade designation PolyActive™.

Biodegradable copolymers having a biodegradable, segmented molecular architecture that includes at least two different ester linkages can also be used. The biodegradable polymers can be block copolymers (of the AB or ABA type) or segmented (also known as multiblock or random-block) copolymers of the (AB), type. These copolymers are formed in a two (or more) stage ring opening copolymerization using two (or more) cyclic ester monomers that form linkages in the copolymer with greatly different susceptibilities to transesterification. Examples of these polymers are described in, for example, in U.S. Pat. No. 5,252,701 (Jarrett et al., “Segmented Absorbable Copolymer”).

Other suitable biodegradable polymer materials include biodegradable terephthalate copolymers that include a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, 6,419,709. Biodegradable terephthalate polyesters can also be used that include a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same). Biodegradable terephthalate polyester can also be used that include a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same). Biodegradable terephthalate polyesters can be used that include a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same).

Biodegradable polyhydric alcohol esters can also be used (See U.S. Pat. No. 6,592,895). This patent describes biodegradable star-shaped polymers that are made by esterifying polyhydric alcohols to provide acyl moieties originating from aliphatic homopolymer or copolymer polyesters. The biodegradable polymer can be a three-dimensional crosslinked polymer network containing hydrophobic and hydrophilic components which forms a hydrogel with a crosslinked polymer structure, such as that described in U.S. Pat. No. 6,583,219. The hydrophobic component is a hydrophobic macromer with unsaturated group terminated ends, and the hydrophilic polymer is a polysaccharide containing hydroxy groups that are reacted with unsaturated group introducing compounds. The components are convertible into a one-phase crosslinked polymer network structure by free radical polymerization. In yet further embodiments, the biodegradable polymer can comprise a polymer based upon α-amino acids (such as elastomeric copolyester amides or copolyester urethanes, as described in U.S. Pat. No. 6,503,538).

In other aspects, a polymeric coating that is associated with the microparticle can control release of polypeptide from microparticles. These aspects will now be described.

The microparticles of the present invention can be immobilized in a polymeric matrix for further release control of the polypeptide. In some aspects, the polymeric matrix can be associated with an implantable medical device, such as in the form of a coating on a surface of the device or a matrix within the device.

The polymeric matrix which entraps the microparticles can be biostable, biodegradable, or can have both biostable and biodegradable properties. The polymeric matrix can be formed from synthetic or natural polymers.

The matrix can be composed of polymeric material (one or more polymers) that allows immobilization of the microparticles. The polymeric material can include one or more homopolymers, copolymers, combinations or blends thereof useful for forming the matrix. Hydrophobic polymers, hydrophilic polymers, or polymers having hydrophobic and hydrophilic properties (such as block or segmented copolymers) can be used to form the matrix. In some cases combinations of polymers having different properties can be used to form the matrix. Hydrophobic polymers are those having no appreciable solubility in water.

Generally, a polymeric material is chosen and used in a composition suitable for forming a matrix with intact microparticles. For example, a polymer can be chosen which is soluble in a liquid that does not destroy the microparticles.

In some modes of practice the polypeptide microparticles are entrapped in a matrix formed from synthetic polymers. Synthetic polymers can be prepared from any suitable monomer including acrylic monomers, vinyl monomers, ether monomers, or combinations of any one or more of these types of monomers. Acrylic monomers include, for example, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, dimethylacrylamide (DMA), and derivatives and/or mixtures of any of these. Vinyl monomers include, for example, vinyl acetate, vinylpyrrolidone, vinyl alcohol, and derivatives of any of these. Ether monomers include, for example, ethylene oxide, propylene oxide, butylene oxide, and derivatives of any of these.

Examples of polymers that can be formed from these monomers include poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, for example, methyl vinyl ether/maleic anhydride copolymers and vinyl pyrrolidone/(meth)acrylamide copolymers. Mixtures of homopolymers and/or copolymers can be used.

In some aspects the first polymer is selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively).

Examples of poly(alkyl(meth)acrylates) include those with alkyl chain lengths from 2 to 8 carbons, inclusive. Exemplary sizes of poly(alkyl(meth)acrylates) are in the range of about 50 kilodaltons to about 1000 kilodaltons, about 100 kilodaltons to about 1000 kilodaltons, about 150 kilodaltons to about 500 kilodaltons, and about 200 kilodaltons to about 400 kilodaltons. One exemplary poly(alkyl(meth)acrylate is poly(n-butyl methacrylate).

Examples of poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly(alkoxyaryl(meth)acrylates).

Some exemplary natural polymers that can be used to form the matrix are low molecular weight starch-derived hydrophobic polymers as described in commonly assigned U.S. patent application Ser. No. 11/724,553 filed on Mar. 15, 2007. (Chudzik et al.). These low molecular weight starch-derived hydrophobic polymers, as exemplified by amylose and maltodextrin, comprise hydrophobic groups and can be used to form hydrophobic matrices that include the polypeptide microparticles.

In some embodiments the polypeptide microparticles are present in a polymeric matrix including a first polymer that is hydrophobic and a second polymer that comprises hydrophobic and hydrophilic portions. Specific examples of such first and second polymers are poly(n-butyl methacrylate) and poly(ethylene glycol) (PEG)/poly(butylene terephthalate) (PBT) block copolymer, respectively (see commonly assigned U.S. Pub. No. 2008/0038354; Slager et al.). In some cases the polymeric matrix can include another (third) polymer that is blendable with the first polymer. A specific examples of a third polymer is poly(ethylene-co-vinyl acetate). The third polymer can be present in the matrix along with the first and second polymer, as a coated layer (e.g., a topcoat) on the polymeric matrix, or both.

The polypeptide microparticles can be associated with a medical device. In some cases, a microparticle-containing coating is formed on the surface of a medical article that is introduced temporarily or permanently into a mammal for the prophylaxis or treatment of a medical condition. These devices include any that are introduced subcutaneously, percutaneously or surgically to rest within an organ, tissue, or lumen of an organ, such as arteries, veins, ventricles, or atria of the heart.

Exemplary medical articles include vascular implants and grafts, grafts, surgical devices; synthetic prostheses; vascular prosthesis including endoprosthesis, stent-graft, and endovascular-stent combinations; small diameter grafts, abdominal aortic aneurysm grafts; wound dressings and wound management device; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices, mitral valve repair devices; left atrial appendage filters; valve annuloplasty devices, catheters; central venous access catheters, vascular access catheters, abscess drainage catheters, drug infusion catheters, parenteral feeding catheters, intravenous catheters (e.g., treated with antithrombotic agents), stroke therapy catheters, blood pressure and stent graft catheters; anastomosis devices and anastomotic closures; aneurysm exclusion devices; biosensors including glucose sensors; cardiac sensors; birth control devices; breast implants; infection control devices; membranes; tissue scaffolds; tissue-related materials; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices, renal devices and hemodialysis devices, colostomy bag attachment devices; biliary drainage products.

In some aspects, a matrix of polymeric material with microparticles, such as a coating, is utilized in connection with an ophthalmic article. The ophthalmic article can be configured for placement at an external or internal site of the eye. In some aspects, the articles can be utilized to deliver a hydrophilic bioactive agent to an anterior segment of the eye (in front of the lens), and/or a posterior segment of the eye (behind the lens). Suitable ophthalmic devices can also be utilized to provide bioactive agent to tissues in proximity to the eye, when desired. Compositions including polymeric material and microparticles can be used either for the formation of a coating on the surface of an ophthalmic article, or in the construction of an ophthalmic article.

Articles configured for placement at an internal site of the eye can reside within any desired area of the eye. In some aspects, the ophthalmic article can be configured for placement at an intraocular site, such as the vitreous. Illustrative intraocular devices include, but are not limited to, those described in U.S. Pat. No. 6,719,750 B2, which describes a non-linear intraocular device.

In other modes of practice, for the construction of an exemplary ophthalmic article for polypeptide release, microparticles can be included in a composition including a biodegradable material, such as a biodegradable polysaccharide as described herein. The composition can be treated to form the article, which can be in a suitable shape, such as a filament, implantation in the eye.

Therapeutic liquid delivery compositions can be prepared that include the polypeptide microparticles. The liquid composition can be prepared for the delivery of the polypeptide microparticles via injection into a target location in the body. For example, the microparticle compositions can be formulated for subcutaneous, intramuscular, and intravenous injections, intrathecal, intraperitoneal, or intraocular injections. If the microparticles do not include a coating or are not encapsulated, the composition is preferably prepared with the microparticles in a non-aqueous composition.

Polypeptides that are released from the microparticles can be used to treat specific diseases. The polypeptide can be used to replace absent or decreased levels of the polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

In some cases the polypeptides of the invention are antibodies or antibody fragments that are used to treat disease, such as those described herein.

A polypeptide can be used to treat or detect hyperproliferative disorders, including neoplasms. A polypeptide released from the microparticles of the present invention may inhibit the proliferation of the disorder through direct or indirect interactions. Alternatively, a polypeptide may cause proliferation of cells, which can inhibit a hyperproliferative disorder. For example, the polypeptide can promote an immune response by causing the proliferation, differentiation, or mobilization of T-cells. This immune response may be increased by either enhancing an existing immune response, or by initiating a new immune response.

Examples of hyperproliferative disorders that can be treated include, but are not limited to neoplasms located in the bone, urogenical tissue, digestive system, liver, pancreas, endocrine glands, eye, nervous system, lymphatic system, spleen, and mammary tissue.

A polypeptide released from the microparticles of the present invention may be used to treat infectious disease. For example, by increasing the immune response, particularly increasing the proliferation and differentiation of B and/or T cells, infectious diseases may be treated. The immune response may be increased by either enhancing an existing immune response, or by initiating a new immune response. Alternatively, the polypeptide may directly inhibit the infectious agent, without necessarily eliciting an immune response.

A polypeptide can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Regeneration also may include angiogenesis.

A polypeptide may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polypeptide may also increase or decrease the differentiation or proliferation of embryonic stem cells.

A polypeptide may be used to modulate mammalian metabolism affecting catabolism, anabolism, processing, utilization, and storage of energy.

In some aspects of the invention, the microparticles are used to treat an ocular disease. The polypeptide microparticles can be used in ocular implants or in association with an implantable ocular device to treat indications such as angiogenesis, inflammation, and degeneration.

For example, the polypeptide microparticles can be used for the treatment of diabetic retinopathy, which is characterized by angiogenesis in the retinal tissue. Diabetic retinopathy has four stages. While the implant can be delivered to a subject diagnosed with diabetic retinopathy during any of these four stages, it is common to treat the condition at a later stage. The polypeptide can be an anti-angiogenic factors used to treat the angiogenesis.

The treatment of diabetic retinopathy can be accomplished by placing the polypeptide microparticles (such as carried by an implant or ocular implantable device) at target location so that anti-angiogenic polypeptide is released and affect the sub-retinal tissue.

In some aspects wherein the microparticle includes a biodegradable material, the invention also provides a method for delivering a polypeptide from a biodegradable microparticle by exposing the microparticle to an enzyme that causes the degradation of the particle. In performing this method a biodegradable microparticle is provided to a subject. The microparticle comprises a natural biodegradable polysaccharide having pendent coupling groups, wherein the microparticle is formed by reaction of the coupling groups to form a crosslinked matrix of a plurality of natural biodegradable polysaccharides, and wherein the microparticle includes a polypeptide. The microparticle is then exposed to a carbohydrase that can promote the degradation of the biodegradable microparticle.

The carbohydrase that contacts the microparticle can specifically degrade the natural biodegradable polysaccharide causing release of the polypeptide. Examples of carbohydrases that can specifically degrade natural biodegradable polysaccharide matrices include α-amylases (which cause the enzymatic degradation of amylose and maltodextrin), such as salivary and pancreatic α-amylases; disaccharidases, such as maltase, lactase and sucrase; trisaccharidases; and glucoamylase (amyloglucosidase).

In some aspects, the carbohydrase can be administered to a subject to increase the local concentration, for example in the tissue or serum surrounding the administered microparticles, so that the carbohydrase may promote the degradation of the microparticles. Exemplary routes for introducing a carbohydrase include local injection, intravenous (IV) routes, and the like. Alternatively, degradation can be promoted by indirectly increasing the concentration of a carbohydrase in the vicinity of the microparticles, for example, by a dietary process, or by ingesting or administering a compound that increases the systemic levels of a carbohydrase.

In other cases, the carbohydrase can be provided in connection with microparticles that are co-administered with the polypeptide microparticles. As the carbohydrase is released from the microparticle, it causes degradation of the matrix and promotes the release of the polypeptide.

The biodegradable polysaccharide compositions are particularly useful for forming biodegradable microparticles that will come in contact with aqueous systems. The body fluids typically have enzymes that allow for the degradation of the natural biodegradable polysaccharide-based particles. The aqueous system (such as bodily fluids) allows for the degradation of the biodegradable composition and release of the polypeptide from the microparticle. In some cases, depending on the polypeptide and the matrix, the polypeptide can diffuse out of the matrix.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

In the Examples, the following assays were utilized for measurement of protein release. ELISA Assay. The elution samples were analyzed for activity of the rabbit antibody molecule using an Enzyme-Linked Immunosorbent Assay (ELISA). Briefly, the wells of 96-well plates were first coated with a goat IgG (Sigma, St. Louis, Mo.; catalog#15256) coating solution, incubated for 90 minutes at room temperature, and then washed 3× with 300 μL PBS/Tween 20 (Sigma). The wells were blocked with 200 μL StabilCoat (SurModics, Eden Prairie, Minn.) for 1 hour at room temperature and then washed 3× with 300 μl PBS/Tween 20. A 100 μl aliquot of elution solution (from the elution of Fab from the polymeric matrices) was added to the appropriate wells and incubated for 1 hour at room temperature, and then washed 3× with PBS/Tween 20. A 100 μL sample of 0.1 g/mL donkey anti-rabbit IgG HRP (Rockland Immunochemicals, Gilbertsville, Pa.; catalog#611-703-127) was added to each well and incubated for 1 hour at room temperature. The wells were washed 4× with 300 μL PBS/Tween 20. A 100 μL of TMB Microwell Peroxidase Substrate System (KPL, catalog#50-76-00; Gaithersburg, Md.) was added to each well. For kinetic assays, the TMB substrate produces a blue color upon reaction with peroxidase. After 15 minutes, the 96-well plate was analyzed for HRP conjugate on a spectrophotometer (Molecular Devices) at 650 nm absorbance. For endpoint analysis, addition of an acidic stop solution will halt color development and turn the TMB substrate yellow. Alternatively, after 15 minutes, 100 μL of a 1N HCl solution was added to the well to stop the reaction. Absorption was then measured at 450 nm. Any variations or modifications to the ELISA Assay are noted in the Examples.

Spectrophotometric Protein Determination. Measurements of protein (Fab fragment) concentration, as eluted from the polymeric matrices of the example, was determined spectrophotometrically by measuring absorbance at about 280 nm (A₂₈₀). Light of this wavelength is absorbed by aromatic amino acids, and most intensely by tryptophan. Calibration samples of Fab fragment were prepared at concentrations 250, 125, 62.5, 31.3, 15.6, and 7.8 μg/mL for preparation of a standard plot. Aliquots of 150 μL of the calibration samples (in triplicate) and 150 μl of elution samples (in duplicate) were pipetted into a black 96-well plate. To all samples 150 μL of a 12 M guanidine-HCl solution in deionized distilled water (DDW) was added. The plate was placed in a freezer and incubated at −20° C. for 10 minutes. After the incubation the 96-well plate was transferred immediately to a plate-reader. λₑₓ=290 nm, λₑₘ=370 nm, cutoff at λ=325 μm.

### Reagents

### Compound I

### APTAC-EITC-Polyethylenimine (APTAC-EITC-PEI) Initiator Polymer

The photoinitiator polymer having pendent photoinitiator groups was prepared as described in Examples 1-2 in U.S. Patent Publication No. 2004/0202774, Chudzik et al., “Charged initiator polymers and methods of use.” An APTAC-EITC-PEI initiator polymer product can be represented by Compound I.

### Compound I

### APTAC-EITC-PEI Initiator Polymer

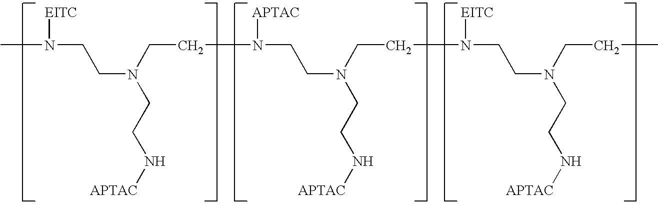

### Compound II

### Maltodextrin-Methacrylate Macromer (MD-Methacrylate)

To provide MD-methacrylate, the following procedure was performed. Maltodextrin (MD; Aldrich; 100 g; 3.67 mmole; Dextrose Equivalent (DE): 4.0-7.0) was dissolved in dimethylsulfoxide (DMSO) 1,000 ml with stirring. The size of the maltodextrin was calculated to be in the range of 2,000 Da to 4,000 Da. Once the reaction solution was complete, 1-methylimidazole (Aldrich; 2.0 g, 1.9 ml) followed by methacrylic-anhydride (Aldrich; 38.5 g) were added with stirring. The reaction mixture was stirred for one hour at room temperature. After this time, the reaction mixture was quenched with water and dialyzed against distilled (DI) water using 1,000 MWCO dialysis tubing. The MD-methacrylate was isolated via lyophilization to give 63.283 g (63% yield). The calculated methacrylate load of macromer was 0.33 μmoles/mg of polymer.

### Compound III

### Maltodextrin-Acrylate Macromer (MD-Acrylate)

### A. Preparation of 3-(acryloyloxy)propanoic acid (2-carboxyethyl acrylate; CEA)

Acrylic acid (100 g; 1.39 mole) and phenothiazine (0.1 g) were placed in a 500 ml round bottom flask. The reaction was stirred at 92° C. for 14 hours. The excess acrylic acid was removed on a rotary evaporator at 25° C. using a mechanical vacuum pump. The amount of residue obtained was 51.3 g. The CEA was used herein without purification.

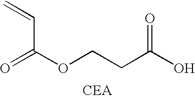

### B. Preparation of 3-chloro-3-oxopropyl acrylate (CEA-Cl)

CEA from above (51 g; ˜0.35 mole) and dimethyl formamide (DMF; 0.2 ml; 0.26 mmole) were dissolved in CH₂Cl₃ (100 ml). The CEA solution was added slowly (over 2 hours) to a stirred solution of oxalyl chloride (53 ml; 0.61 mole), DMF (0.2 ml; 2.6 mmole), anthraquinone (0.5 g; 2.4 mmole), phenothiazine (0.1 g, 0.5 mmole), and CH₂Cl₃ (75 ml) in a 500 ml round bottom flask in an ice bath at 200 mm pressure. A dry ice condenser was used to retain the CH₂Cl₃ in the reaction flask. After the addition was complete the reaction was stirred at room temperature overnight. The weight of reaction solution was 369 g. A sample of the CEA-Cl reaction solution (124 mg) was treated with 1,4-dibromobenzene (DBB, 6.85 mg) evaporated and dissolved in CDCl₃: ¹H NMR (CDCl₃, 400 MHz) δ 7.38 (s, 4H; DBB internal std.), 6.45 (d, 1H, J=17.4 Hz), 6.13 (dd, 1H, J=17.4, 10.4 Hz), 5.90 (d, 1H, J=10.4 Hz), 4.47 (t, 2H, J=5.9 Hz), 3.28 (t, 2H, J=5.9). The spectra were consistent with the desired product. There was 0.394 mole DBB for 1.0 mole CEA-Cl by integration, which gave a calculated yield of 61%. Commercially available CEA (426 g; Aldrich) was reacted with oxalyl chloride (532 ml) in a procedure similar to the one listed above. The residue CEA-Cl (490 g) was distilled using an oil bath at 140° C. at a pressure of 18 mm Hg. The distillate temperature reached 98° C. and 150 g of distillate was collected. The distillate was redistilled at 18 mm Hg at a maximum bath temperature of 120° C. The temperature range for the distillate was 30° C. to 70° C., which gave 11 g of material. The distillate appeared to be 3-chloro-3-oxopropyl 3-chloropropanoate. The residue of the second distillation (125 g; 26% of theory) was used in step C below.

### C. Preparation of 3-azido-3-oxopropyl acrylate (CEA-N3)

CEA-Cl from step B (109.2 g; 0.671 mole) was dissolved in acetone (135 ml). Sodium azide (57.2 g; 0.806 mole) was dissolved in water (135 ml) and chilled. The CEA-Cl solution was then added to the chilled azide solution with vigorous stirring in an ice bath for 1.5 hours. The reaction mixture was extracted two times with 150 ml of CHCl₃ each extraction. The CHCl₃ solution was passed through a silica gel column 40 mm in diameter by 127 mm. The 3-azido-3-oxopropyl acrylate solution was gently agitated over dried molecular sieves at 4° C. overnight. The dried solution was used in step D without purification.

### D. Preparation of 2-isocyanatoethyl acrylate (EA-NCO)

The dried azide solution (from step C) was slowly added to refluxing CHCl₃, 75 ml. After the addition was completed, refluxing was continued 2 hours. The EA-NCO solution (594.3 g) was protected from moisture. A sample of the EA-NCO solution (283.4 mg) was mixed with DBB (8.6 mg) and evaporated. The residue was dissolved in CDCl₃: ¹H NMR (CDCl₃, 400 MHz) δ 7.38 (s, 4H; DBB internal std.), 6.50 (d, 1H, J=17.3 Hz), 6.19 (dd, 1H, J=17.3, 10.5 Hz), 5.93 (d, 1H, J=10.5 Hz), 4.32 (t, 2H, J=5.3 Hz), 3.59 (t, 2H, J=5.3). The spectra were consistent with the desired EA-NCO. There was 0.165 mole DBB for 1.0 mole EA-NCO by integration, which gave a calculated concentration of 110 mg EA-NCO/g of solution. The EA-NCO solution was used to prepare a macromer in step E.

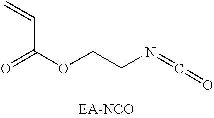

### E. Preparation of Maltodextrin-acrylate macromer (MD-Acrylate) (Compound III)

Maltodextrin (MD; Aldrich; 9.64 g; approximately 3.21 mmole; DE: 4.0-7.0) was dissolved in dimethylsulfoxide (DMSO) 60 ml. The size of the maltodextrin was calculated to be in the range of 2,000 Da-4,000 Da. A solution of EA-NCO from step D (24.73 g; 19.3 mmole) was evaporated and dissolved in dried DMSO (7.5 ml). The two DMSO solutions were mixed and heated to 55° C. overnight. The DMSO solution was placed in dialysis tubing (1000 MWCO, 45 mm flat width×50 cm long) and dialyzed against water for 3 days. The macromer solution was filtered and lyophilized to give 7.91 g white solid. A sample of the macromer (49 mg), and DBB (4.84 mg) was dissolved in 0.8 ml DMSO-d₆: ¹H NMR (DMSO-d₆, 400 MHz) δ 7.38 (s, 4H; internal std. integral value of 2.7815), 6.50, 6.19, and 5.93 (doublets, 3H; vinyl protons integral value of 3.0696). The calculated acrylate load of macromer was 0.616 μmoles/mg of polymer.

### Compound IV

### Polyacrylamide Polymer Having Photoreactive Groups and Thermally Reactive Groups

### A. Preparation of 4-Benzoylbenzoyl Chloride (BBA-Cl)

The compound 4-Benzoylbenzoic acid (BBA), 1.0 kg (4.42 moles), was added to a dry 5 liter Morton flask equipped with reflux condenser and overhead stirrer, followed by the addition of 645 ml (8.84 moles) of thionyl chloride and 725 ml of toluene. Dimethylformamide, 3.5 ml, was then added and the mixture was heated at reflux for 4 hours. After cooling, the solvents were removed under reduced pressure and the residual thionyl chloride was removed by three evaporations using 3×500 ml of toluene. The product was recrystallized from 1:4 toluene:hexane to give 988 g (91% yield) after drying in a vacuum oven. Product melting point was 92-94° C. Nuclear magnetic resonance (NMR) analysis (¹H NMR (CDCl₃)) was consistent with the desired product: aromatic protons 7.20-8.25 (m, 9H). All chemical shift values are in ppm downfield from a tetramethylsilane internal standard. The final compound was stored for use in the preparation of a monomer used in the synthesis of APMA-HCl below.

### B. Preparation of N-(3-Aminopropyl)methacrylamide Hydrochloride (APMA-HCl)

A solution of 1,3-diaminopropane, 1910 g (25.77 moles), in 1000 ml of CH₂Cl₂ was added to a 12 liter Morton flask and cooled on an ice bath. A solution of t-butyl phenyl carbonate, 1000 g (5.15 moles), in 250 ml of CH₂Cl₂ was then added dropwise at a rate which kept the reaction temperature below 15° C. Following the addition, the mixture was warmed to room temperature and stirred for 2 hours. The reaction mixture was diluted with 900 ml of CH₂Cl₂ and 500 g of ice, followed by the slow addition of 2500 ml of 2.2 N NaOH. After testing to insure the solution was basic, the product was transferred to a separatory funnel and the organic layer was removed and set aside as extract #1. The aqueous portion was then extracted with 3×1250 ml of CH₂Cl₂, keeping each extraction as a separate fraction. The four organic extracts were then washed successively with a single 1250 ml portion of 0.6 N NaOH beginning with fraction #1 and proceeding through fraction #4. This wash procedure was repeated a second time with a fresh 1250 ml portion of 0.6 N NaOH. The organic extracts were then combined and dried over Na₂SO₄. Filtration and evaporation of solvent to a constant weight gave 825 g of N-mono-t-BOC-1,3-diaminopropane which was used without further purification.

A solution of methacrylic anhydride, 806 g (5.23 moles), in 1020 ml of CHCl₃ was placed in a 12 liter Morton flask equipped with overhead stirrer and cooled on an ice bath. Phenothiazine, 60 mg, was added as an inhibitor, followed by the dropwise addition of N-mono-t-BOC-1,3-diaminopropane, 825 g (4.73 moles), in 825 ml of CHCl₃. The rate of addition was controlled to keep the reaction temperature below 10° C. at all times. After the addition was complete, the ice bath was removed and the mixture was left to stir overnight. The product was diluted with 2400 ml of water and transferred to a separatory funnel. After thorough mixing, the aqueous layer was removed and the organic layer was washed with 2400 ml of 2 N NaOH, insuring that the aqueous layer was basic. The organic layer was then dried over Na₂SO₄ and filtered to remove drying agent. A portion of the CHCl₃ solvent was removed under reduced pressure until the combined weight of the product and solvent was approximately 3000 g. The desired product was then precipitated by slow addition of 11.0 liters of hexane to the stirred CHCl₃ solution, followed by overnight storage at 4° C. The product was isolated by filtration and the solid was rinsed twice with a solvent combination of 900 ml of hexane and 150 ml of CHCl₃. Thorough drying of the solid gave 900 g of N-[3-(N-tert-butyloxycarbonylamino)-propyl]-methacrylamide, m.p. 85.8° C. by DSC (Differential Scanning Calorimeter). Analysis on an NMR spectrometer was consistent with the desired product: ¹H NMR (CDCl₃) amide NH's 6.30-6.80, 4.55-5.10 (m, 2H), vinyl protons 5.65, 5.20 (m, 2H), methylenes adjacent to N 2.90-3.45 (m, 4H), methyl 1.95 (m, 3H), remaining methylene 1.50-1.90 (m, 2H), and t-butyl 1.40 (s, 9H).

A 3-neck, 2 liter round bottom flask was equipped with an overhead stirrer and gas sparge tube. Methanol, 700 ml, was added to the flask and cooled on an ice bath. While stirring, HCl gas was bubbled into the solvent at a rate of approximately 5 liters/minute for a total of 40 minutes. The molarity of the final HCl/MeOH solution was determined to be 8.5 M by titration with 1 N NaOH using phenolphthalein as an indicator. The N-[3-(N-tert-butyloxycarbonylamino)-propyl]-methacrylamide, 900 g (3.71 moles), was added to a 5 liter Morton flask equipped with an overhead stirrer and gas outlet adapter, followed by the addition of 1150 ml of methanol solvent. Some solids remained in the flask with this solvent volume. Phenothiazine, 30 mg, was added as an inhibitor, followed by the addition of 655 ml (5.57 moles) of the 8.5 M HCl/MeOH solution. The solids slowly dissolved with the evolution of gas but the reaction was not exothermic. The mixture was stirred overnight at room temperature to insure complete reaction. Any solids were then removed by filtration and an additional 30 mg of phenothiazine were added. The solvent was then stripped under reduced pressure and the resulting solid residue was azeotroped with 3×1000 ml of isopropanol with evaporation under reduced pressure. Finally, the product was dissolved in 2000 ml of refluxing isopropanol and 4000 ml of ethyl acetate were added slowly with stirring. The mixture was allowed to cool slowly and was stored at 4° C. overnight. The APMA-HCl was isolated by filtration and was dried to constant weight, giving a yield of 630 g with a melting point of 124.7° C. by DSC. Analysis on an NMR spectrometer was consistent with the desired product: ¹H NMR (D₂O) vinyl protons 5.60, 5.30 (m, 2H), methylene adjacent to amide N 3.30 (t, 2H), methylene adjacent to amine N 2.95 (t, 2H), methyl 1.90 (m, 3H), and remaining methylene 1.65-2.10 (m, 2H). The final compound was stored for use in the preparation of BBA-APMA below.

### C. Preparation of N-[3-(4-Benzoylbenzamido)propyl]methacrylamide (BBA-APMA)

APMA-HCl, 120 g (0.672 moles), prepared according to the general method described above, was added to a dry 2 liter, three-neck round bottom flask equipped with an overhead stirrer. Phenothiazine, 23-25 mg, was added as an inhibitor, followed by 800 ml of chloroform. The suspension was cooled below 10° C. on an ice bath and 172.5 g (0.705 moles) of BBA-Cl, prepared according to the general method described above, were added as a solid. Triethylamine, 207 ml (1.485 moles), in 50 ml of chloroform was then added dropwise over a 1-1.5 hour time period. The ice bath was removed and stirring at ambient temperature was continued for 2.5 hours. The product was then washed with 600 ml of 0.3 N HCl and 2×300 ml of 0.07 N HCl. After drying over sodium sulfate, the chloroform was removed under reduced pressure and the product was recrystallized twice from 4:1 toluene:chloroform using 23-25 mg of phenothiazine in each recrystallization to prevent polymerization. Typical yields of BBA-APMA were 90% with a melting point of 147-151° C. Analysis on an NMR spectrometer was consistent with the desired product: ¹H NMR (CDCl₃) aromatic protons 7.20-7.95 (m, 9H), amide NH 6.55 (broad t, 1H), vinyl protons 5.65, 5.25 (m, 2II), methylene adjacent to amide N's 3.20-3.60 (m, 4H), methyl 1.95 (s, 3H), and remaining methylene 1.50-2.00 (m, 2H). The final compound was stored for use in the synthesis of photoactivatable polymers as described below.

### D. Preparation of N-Succinimidyl 6-Maleimidohexanoate (MAL-EAC-NOS)

A functionalized monomer was prepared in the following manner, and was used as described herein to introduce activated ester groups on the backbone of a polymer. 6-Aminohexanoic acid, 100 g (0.762 moles), was dissolved in 300 ml of acetic acid in a three-neck, 3 liter flask equipped with an overhead stirrer and drying tube. Maleic anhydride, 78.5 g (0.801 moles), was dissolved in 200 ml of acetic acid and added to the 6-aminohexanoic acid solution. The mixture was stirred one hour while heating on a boiling water bath, resulting in the formation of a white solid. After cooling overnight at room temperature, the solid was collected by filtration and rinsed with 2×50 ml of hexane. After drying, the typical yield of the (Z)-4-oxo-5-azo-2-undecendioic acid was 158-165 g (90-95%) with a melting point of 160-165° C. Analysis on an NMR spectrometer was consistent with the desired product: ¹H NMR (DMSO-d₆) amide proton 8.65-9.05 (m, 1H), vinyl protons 6.10, 6.30 (d, 2H), methylene adjacent to nitrogen 2.85-3.25 (m, 2H), methylene adjacent to carbonyl 2.15 (t, 2H), and remaining methylenes 1.00-1.75 (m, 6H).

(Z)-4-oxo-5-azo-2-undecendioic acid, 150.0 g (0.654 moles), acetic anhydride, 68 ml (73.5 g, 0.721 moles), and phenothiazine, 500 mg, were added to a 2 liter three-neck round bottom flask equipped with an overhead stirrer. Triethylamine, 91 ml (0.653 moles), and 600 ml of tetrahydrofuran (THF) were added and the mixture was heated to reflux while stirring. After a total of 4 hours of reflux, the dark mixture was cooled to about 60° C. and poured into a solution of 250 ml of 12 N HCl in 3 liters of water. The mixture was stirred 3 hours at room temperature and then was filtered through a filtration pad (Celite 545, J. T. Baker, Jackson, Term.) to remove solids. The filtrate was extracted with 4×500 ml of chloroform and the combined extracts were dried over sodium sulfate. After adding 15 mg of phenothiazine to prevent polymerization, the solvent was removed under reduced pressure. The 6-maleimidohexanoic acid was recrystallized from 2:1 hexane:chloroform to give typical yields of 76-83 g (55-60%) with a melting point of 81-85° C. Analysis on a NMR spectrometer was consistent with the desired product: ¹H NMR (CDCl₃) maleimide protons 6.55 (s, 2H), methylene adjacent to nitrogen 3.40 (t, 2H), methylene adjacent to carbonyl 2.30 (t, 2H), and remaining methylenes 1.05-1.85 (m, 6H).

The 6-maleimidohexanoic acid, 20.0 g (94.7 mmol), was dissolved in 100 ml of chloroform under an argon atmosphere, followed by the addition of 41 ml (0.47 mol) of oxalyl chloride. After stirring for 2 hours at room temperature, the solvent was removed under reduced pressure with 4×25 ml of additional chloroform used to remove the last of the excess oxalyl chloride. The acid chloride was dissolved in 100 ml of chloroform, followed by the addition of 12 g (0.104 mol) of N-hydroxysuccinimide and 16 ml (0.114 mol) of triethylamine. After stirring overnight at room temperature, the product was washed with 4×100 ml of water and dried over sodium sulfate. Removal of solvent gave 24 g of product (82%), which was used without further purification. Analysis on an NMR spectrometer was consistent with the desired product: ¹H NMR (CDCl₃) maleimide protons 6.60 (s, 2H), methylene adjacent to nitrogen 3.45 (t, 2H), succinimidyl protons 2.80 (s, 4H), methylene adjacent to carbonyl 2.55 (t, 2H), and remaining methylenes 1.15-2.00 (m, 6H). The final compound was stored for use in the synthesis of photoactivatable polymers as described herein.

### E. Preparation of Copolymer of N,N-dimethylacrylamide (DMA), BBA-APMA, and MAL-EAC-NOS (Photo DMA-NOS) (Compound IV) (Azo)

A photoactivatable copolymer was prepared in the following manner. N,N-dimethylacrylamide (DMA, 41.46 g (419 mmol)), BBA-APMA, prepared according to the general method described herein, 1.56 g (4.5 mmol), Compound MAL-EAC-NOS, prepared according to the general method described herein, 6.88 g (22.3 mmol), and azobis(2-methyl-butyronitrile) (Vazo-67) 1.4 g (7.3 mmol) were dissolved in 200 ml of tetrahydrofuran (THF). The THF solution was added to a second stirred refluxing solution of Vazo 67 0.34 g (1.8 mmol) in THF (50 ml) under an inert atmosphere over one hour. The solution was refluxed overnight with stirring under an inert atmosphere. The polymer was isolated by slow addition of the THF solution to vigorously stirred hexanes (2500 ml). The precipitated polymer product was isolated by filtration and the filter cake was rinsed thoroughly with 200 ml hexanes. The product was dried under vacuum at 30° C. to give 51.7 g of a white solid.

### Compound V

### Ethylenebis(4-benzoylbenzyldimethylammonium)Dibromide (Diphoto-Diquat) (TEMED-DQ)

N,N,N′,N′-Tetramethylethylenediamine, 6 g (51.7 mmol), was dissolved in 225 ml of chloroform with stirring. 4-Bromomethylbenzophenone, 29.15 g (106.0 mmol), was added as a solid and the reaction mixture was stirred at room temperature for 72 hours. After this time, the resulting solid was isolated by filtration and the white solid was rinsed with cold chloroform. The residual solvent was removed under vacuum and 34.4 g of solid were isolated for a 99.7% yield, melting point 218-220° C. Analysis on an NMR spectrometer was consistent with the desired product: ¹H NMR (DMSO-d₆) aromatic protons 7.20-7.80 (m, 18H), benzylic methylenes 4.80 (br. s, 4H), amine methylenes 4.15 (br. s, 4H), and methyls 3.15 (br. s, 12H).

### Compound V

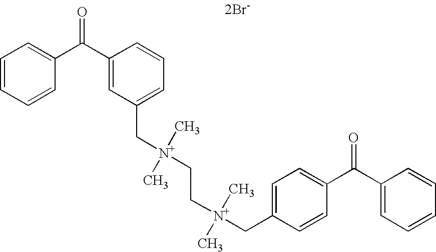

### Compound VI

### 4,5-bis(4-benzoylphenylmethyleneoxy)benzene-1,3-disulfonic acid disodium salt (DBDS)

The initiator 4,5-bis(4-benzoylphenylmethyleneoxy)benzene-1,3-disulfonic acid disodium salt (DBDS) was prepared as follows. An amount (9.0 g, 0.027 moles) of 4,5-dihydroxy 1,3-benzene disulfonic acid disodium salt monohydrate was added to a 250 ml, 3 necked round bottom flask fitted with an overhead stirrer, gas inlet port, and reflux condenser. An amount (15 g, 0.054 moles) of 4-bromomethylbenzophenone (BMBP), 54 ml tetrahydrofuran (THF), and 42 ml deionized water were then added. The flask was heated with stirring under an argon atmosphere to reflux. The argon atmosphere was maintained during the entire time of refluxing.

After reflux was reached, 9.0 ml (6 N, 0.054 moles) of a sodium hydroxide solution was added through the reflux condenser. The reaction was stirred under reflux for 3 hours. After this time, a second portion of BMBP, 3.76 g (0.014 moles), and 3.6 ml (6 N, 0.022 moles) of sodium hydroxide were added. The reaction was continued under reflux for more than 12 hours, after the second BMBP addition.

The reaction mixture was evaporated at 40° C. under vacuum on a rotary evaporator to give 46 g of a yellow paste. The paste was extracted by suspending three times in 50 ml of chloroform at 40° C. for 30 minutes. A centrifuge was used to aid in the decanting of the chloroform from the solid. The solid was collected on a Büchner funnel, after the last extraction, and air dried for 30 minutes. The solid was then dried by using a rotary evaporator with a bath temperature of 50° C. at a pressure of about 1 mm for 30 minutes. The dried solid, 26.8 g, was recrystallized from 67 ml of water and 67 ml of methanol. The dried purified product amounted to 10.4 g (the theoretical yield was 19.0 g) with absorbance of 1.62 at 265 nm for a concentration of 0.036 mg/ml.

### Compound VII

### Polyalditol-Acrylate

Polyalditol (PA; GPC; 9.64 g; ˜3.21 mmole) was dissolved in dimethylsulfoxide (DMSO) 60 ml. The size of the polyalditol was calculated to be in the range of 2,000 Da-4,000 Da. A solution of EA-NCO as described herein (24.73 g; 19.3 mmole) was evaporated and dissolved in dried DMSO (7.5 ml). The two DMSO solutions were mixed and heated to 55° C. overnight. The DMSO solution was placed in dialysis tubing (1000 MWCO, 45 mm flat width×50 cm long) and dialyzed against water for 3 days. The polyalditol macromer solution was filtered and lyophilized to give 7.91 g white solid. A sample of the macromer (49 mg), and DBB (4.84 mg) was dissolved in 0.8 ml DMSO-d₆: ¹H NMR (DMSO-d₆, 400 MHz) δ 7.38 (s, 4H; internal std. integral value of 2.7815), 6.50, 6.19, and 5.93 (doublets, 3H; vinyl protons integral value of 3.0696). The calculated acrylate load of macromer was 0.616 μmoles/mg of polymer.

### Compound VIII

### Poly(Ethylene Glycol)-di(Imidazolyl Carbonate)

Compound VIII was synthesized as described in co-pending application Ser. No. 11/789,786, filed Apr. 25, 2007, Jelle et al. (see Example 7).

Poly(ethylene glycol), M_{w} 600, (30.15 g) was transferred to a 150 ml round bottom flask and dissolved with 50 ml dichloromethane (DCM). The solvent was stripped off using a rotary evaporator and high temperature water bath. This step was repeated twice more. In a 500 ml round bottom flask 1,1′-carbonyldiimidazole, CDI, (22.90 g) was dissolved with 250 ml DCM. A Teflon stir bar was inserted into the CDI solution and placed on a stir plate under nitrogen. The PEG₆₀₀ was dissolved with 50 ml DCM and slowly added to the stirring CDI solution and stirred at room temperature for two hours under nitrogen. The reaction solution was transferred into a 1 L separatory funnel and washed twice with 1 mM HCl followed by two brine solution washes. The organic solution was collected and dried with magnesium sulfate. The dried solution was filtered through a Whatman paper filter into a clean 500 ml round bottom flask and the DCM was rotary evaporated with mild heat (30° C.). A clear, slightly yellowish-tinted oil was collected (37.02 g).

### Other Materials

In some aspects the polymer that can be blended with the first polymer is poly(ethylene-co-vinyl acetate). For example, the blend can be a combination of poly(n-butyl methacrylate) (pBMA) and poly(ethylene-co-vinyl acetate) (pEVA). Such blends are described in commonly assigned U.S. Pat. No. 6,214,901 (Chudzik et al.) and US Publication No. 2002/0188037 A1 (Chudzik et al.).

using pEVA (33 weight percent vinyl acetate; Aldrich Chemical, Milwaukee, Wis.) and pBMA (337,000 average molecular weight; Aldrich Chemical, Milwaukee, Wis.)

The polymer PEG₁₀₀₀-45PBT-55 is a copolymer of poly(butyleneterephthalate-co-ethylene glycol) copolymer with 45 wt. % polyethylene glycol having an average molecular weight of 1000 kD and 55 wt. % butyleneterephthalate. PEG₁₀₀₀-45PBT-55 is commercially available from OctoPlus (Leiden, Netherlands) under the product name PolyActive™.

The macromer “MD-acrylate” is an acrylated maltodextrin polymer prepared as described in U.S. Patent Publication No. 2007/0065481.

Polyvinyl pyrrolidone (PVP) Kollidion 90F was obtained from BASF Mt. Olive, N.J. (cat #85-2549). Poly(ethylene glycol) (PEG) was obtained from Union Carbide, Danbury, Conn. (#37255-26-6).

The photo-reagent 4,5-bis(4-benzoylphenylmethyleneoxy)benzene-1,3-disulfonic acid disodium salt (DBDS) was prepared as described in Example 1 of U.S. Pat. No. 6,669,994.

Colloidal Gold 5 nm, 0.01% w/v, 5 μg gold, 0.00013% w/w protein, was purchased from VWR, West Chester, Pa. (cat#IC15401005).

Spray coating was performed using an Ultrasonic Spray Coater as described U.S. Published Application 2004/0062875, or an IVEK Coater having asyringe pump connected to an IVEK gas atomization spray system (DIgispense 2000 Model #4065, IVEK, North Springfield, Vt.) as described in U.S. Published Application 2005/0244453.

### EXAMPLE 1

### Formation of Fab Microparticles with Coatings

This series of experiments studied various microparticle coating compositions on colloidal gold microparticles. A 5 mM PBS solution without NaCl was prepared from a 10×PBS stock solution. The PBS was diluted in DDW to a total volume of 500 ml. The pH was adjusted to 7.31 after adding one drop of H₃PO₄.

1A. Preparation of Fab Microparticles with Colloidal Gold.

Fab (rabbit anti-goat (RαG)) was desalted using a BioRad desalting column (Econo-Pac™ 10 DG). Storage buffer from the columns was disposed. The columns were eluted with 20 mL of 5 mM PBS as prepared above. An amount of Fab (RαG), 2.5 mL, (A₂₈₀(50 μL)=0.953, ε=1.35=>14.1 mg/mL) was put on each column and allowed to completely absorb. Fab was eluted from the columns with 4 ml of 5 mM PBS.

The Fab was then concentrated using four centrifuge filters (10 kDa cutoff, PALL LifeSciences), which were filled with 4 mL of the desalted Fab eluate and spun at 5500 g for 50 minutes at 10° C. The concentrated Fab supernatants were combined providing Fab at a concentration of 20.4 mg/ml as determined spectrophotometrically (A₂₈₀). The pH of the protein solution was adjusted to 5.3 by adding 10 μL of 2N HCl.

To 2 mL of the concentrated Fab protein (40 mg), 50 μL colloidal gold (VWR, 5 nm, 0.01% w/v, 5 μg gold, 0.00013% w/w protein) solution was added. The protein/colloidal gold solution was incubated at 50° C. for 40 minutes in a 15 mL centrifuge tube.

A PEG solution (20 kDa dissolved to 30% w/v in DI water, pH=5) was warmed to 50° C. A hole was drilled in the screw-cap of the centrifuge tube containing the Fab protein/colloidal gold solution, and 700 μL of the PEG solution, 5.25× protein weight, was added to the protein/colloidal gold solution while vortexing thoroughly during the addition and another five (5) seconds thereafter.

A slightly turbid solution was obtained and poured in a plastic Petri-dish. The dish was covered and placed at −20° C. for 1.5 hour, and then on dry ice for 30 minutes. The initially glossy appearance of the PEG/protein suspension became matted and solid. The frozen suspension was then lyophilized in a vacuum oven at room temperature over night.

Following lyophilization, PEG was extracted using chloroform. Once no soft spots were observed, the dry cake was transferred to a 50 mL centrifuge tube. A 20 mL aliquot of chloroform was added. The PEG dissolved, rendering a cloudy fine protein suspension. The chloroform was dispensed in 4 PTFE filters 0.2 um (Amicon, Ultrafree™-CL) and centrifuged at 5500 rpm, 10° C. for 15 minutes. Using glass pipettes, fresh chloroform was added. This washing procedure was done 3 times in total. The protein particles were resuspended in 10 mL chloroform.

The colloidal gold-Fab microparticles were utilized in the following Examples 1B-1D.

### 1B. Compound I (PEI-APTAC-EITC) Microparticle Coating

Batches of the prepared colloidal gold-Fab microparticles were prepared by suspending 4 mg of the microparticles in 1 mL chloroform. The suspensions were placed in centrifuge tubes. To the particles, 10, 25, 50 or 100 μL of a solution containing 2 mg/mL of Compound I (PEI-APTAC-EITC) in methanol (MeOH) was added. Appropriate amounts of methanol were added to obtain a 10:1 chloroform/methanol mixture in each of the samples. The mixtures were incubated at room temperature for 20 minutes. The solutions became colorless and the particles were visibly coated with Compound I.

Any excess Compound I (PEI-APTAC-EITC) was removed by spinning the particles in PTFE filters (0.2 μm (Amicon, Ultrafree™-CL)) at 3000 rpm for 3 minutes. Particles were then rinsed using CHCl₃ and spun again at 3000 rpm for 3 minutes.

Next, solvent was removed from the particles by drying them in a vacuum oven. Release of polypeptide from the coated microparticles was then performed in PBS. (Particles were found insoluble when suspended in PBS; however, over the course of 24 to 48 hours, the suspension of coated microparticles slowly dissolved.) Without a particle coating, the particles rapidly dissolved in PBS within about 10 seconds.

The coated microparticles were suspended in 1 mL regular PBS (0.01 M) in microcentrifuge tubes. At specific time intervals the coated microparticles were spun down at 5000 rpm for 5 minutes. The elution medium was removed and analyzed, and then the particles were resuspended in fresh PBS. The elution medium was assayed for Fab release, utilizing an ELISA assay and spectrophotometric protein determination. Results are summarized in Table 2. The numbers in the table represent the concentration of Fab in mg/mL in 1 mL elution medium, with a total of 5 mg of Fab used in elution studies.

TABLE 2
Release of Fab from microparticles.

Compound I (2 mg/mL)

| Time (hours) | 10 μL | 25 μL | 50 μL | 100 μL |
|---|---|---|---|---|
| 1 | 4032.5 | 4235.6 | 4689.0 | 3576.8 |
| 4 | 5.4 | 7.7 | 2.8 | 14.0 |
| 24 | 3.1 | 2.6 | 2.7 | 23.9 |
| Total | 4041.1 | 4245.9 | 4694.6 | 3614.6 |

1B(2). Device Polymer Coating Including Polypeptide Microparticles with Microparticle Coating

A batch of the prepared colloidal gold-Fab microparticles as prepared in 1A was in an amount of 50 mg was suspended in 5 mL chloroform. The suspension was placed in centrifuge tubes. To the particles, 500 μL of a solution containing 2 mg/mL Compound I (PEI-APTAC-EITC) in methanol (MeOH) was added. An appropriate amount of chloroform was added to obtain a 10:1 chloroform/methanol mixture. The mixture was incubated at room temperature for 20 minutes. The solutions became colorless and the particles were visibly coated with Compound I as determined by the EITC color, viewed with bright and dark field microscopy.

Excess Compound I (PEI-APTAC-EITC) was removed by spinning the particles in PTFE filters (0.2 μm (Amicon, Ultrafree-CL)) at 3000 rpm for 3 minutes. Particles were then rinsed using CHCl₃ and spinning again at 3000 rpm for 3 minutes.

For the preparation of a device coating composition, the coated microparticles were resuspended in chloroform, by adding a solution containing 20 mg/mL pBMA, 20 mg/mL pEVA, and 11 mg/mL 1000PEG₄₅PBT₅₅. Particles were mixed with coating solution at 30% w/w concentrations. Eight helical intravitreal implants constructed from MP-35 alloy (see commonly assigned U.S. Pub. No. 2005/0019371) were coated per formulation, with a target coating weight of 1 mg coated solid material per coil using an ultrasonic spray coating method as described herein. Four of the coated intravitreal implants were subsequently topcoated with a 20 mg/mL pEVA (33% vinyl acetate content)_solution in chloroform, aiming for 300 ug topcoat weight, coated also using the ultrasonic coating apparatus Results are shown in Table 3 and FIG. 1, with time (days) x-axis and Cumulative release (%) (calculated based on theoretical total amount of protein in coating on y-axis). The control was a pEVA/pBMA/000PEG₄₅PBT₅₅ coating without the compound I coating on the microparticles. For the elution studies, intravitreal implants were placed in deepwell plates with 1 mL PBS buffer at 37° C.

TABLE 3

| | | | | particle coating with |
|---|---|---|---|---|
| time | | PEVA | particle coating | compound I and |
| (days) | control | topcoat | with compound I | PEVA topcoat |
| 0.1 | 27.39192 | 0.934466 | 5.104123 | 0.072735 |
| 1 | 92.83765 | 11.48654 | 12.4303 | 0.693679 |
| 3 | 106.4074 | 37.44785 | 25.84595 | 4.815132 |
| 4 | 106.847 | 41.17399 | 31.08446 | 5.653602 |
| 7 | 107.2764 | 60.37707 | 42.68506 | 6.567052 |
| 10 | 107.5789 | 72.714 | 45.96734 | 7.002361 |
| 14 | 107.8922 | 86.49505 | 50.54089 | 7.751431 |
| 18 | 108.0396 | 89.87339 | 52.22989 | 9.881504 |
| 21 | 108.1435 | 92.34432 | 52.88845 | 10.39357 |
| 24 | 108.6943 | 94.20921 | 54.10525 | 10.90131 |
| 28 | 108.7418 | 95.64736 | 54.69086 | 13.25787 |
| 32 | 108.7759 | 96.70519 | 55.14622 | 13.479 |

### EXAMPLE 1B(3)

### Use of PEI or Compound I as Additive to Coating

Spray-dried Fab (non-specific) particles (70% Fab/30% trehalose) were used, made by Brookwood Laboratories

For the preparation of a device coating composition, the formulations as described in Table 4 were prepared in 5 mL of chloroform with 25 mg Fab particles, 40% w/w of the total formulation, and a mixture of 1000PEG₄₅PBT₅₅ and pEVA polymers. PEI and Compound I were added last to the formulations.

TABLE 4

| | Components of coating composition | | | | | | | |
|---|---|---|---|---|---|---|---|---|
| | Protein | | | | | | | |
| | (from | | 1000PEG₅₅PBT₄₅ | | pEVA | | PEI or Compound I | |
| | microparticles) | | | mL | | mL | | mL |
| | % | mg | % | (at 40 mg/ml) | % | (at 40 mg/ml) | % | (at 10 mg/ml) |
| control | 40 | 25 | 50.0 | 0.78 | 10.00 | 0.16 | — | — |
| PEI 2% | 40 | 25 | 48.3 | 0.76 | 9.67 | 0.15 | 2.00 | 0.13 |
| PEI 10% | 40 | 25 | 41.7 | 0.65 | 8.33 | 0.13 | 10.00 | 0.63 |
| Cpd I 2% | 40 | 25 | 48.3 | 0.76 | 9.67 | 0.15 | 2.00 | 0.13 |
| Cpd I 10% | 40 | 25 | 41.7 | 0.65 | 8.33 | 0.13 | 10.00 | 0.63 |

Four intravitreal implants were coated per formulation and coated as described in Example 1B(2). The coated intravitreal implants were dried in a nitrogen box overnight and put for release in 1 ml PBS as described in Example 1B(2). Protein concentration was determined using tryptophan assay: 100 uL of calibration solutions and of the release samples were pipetted in a 96-blackwell plate and mixed with 100 ul of a 12 M Guanidine.HCl solution in DDW. The plate was incubated for 10 minutes at −20° C. and immediately analyzed using a plate reader equipped with fluorescence detector. λₑₓ=290, λₑₘ=370, λ_{cutoff}=325. Results of Fab release are shown in Table 5 and FIG. 2.

TABLE 5

| | | Com- | PEI 25 | | | | |
|---|---|---|---|---|---|---|---|
| | | pound I | kDa | | | | |
| time | Control | 2% | 2% | PEI 25 kDa 10% | | Ir01 10% | |
| (days) | Aver | Aver | Aver | Aver | SD | Aver | SD |
| 0 | 0 | 0 | 0 | 0 | 0 | 0 | 0 |
| 0.1 | 8.7 | 3.5 | 3.3 | 6.85 | 2.21 | 13.86 | 2.60 |
| 0.8 | 15.4 | 6.7 | 5.5 | 14.98 | 3.34 | 20.14 | 2.90 |
| 2.3 | 19.3 | 9.2 | 7.2 | 21.34 | 3.31 | 23.29 | 3.18 |
| 3.8 | 21.5 | 11.2 | 8.7 | 25.20 | 3.05 | 24.95 | 3.33 |
| 6 | 22.1 | 12.1 | 9.3 | 27.90 | 2.68 | 25.40 | 3.41 |
| 11 | 23.7 | 13.9 | 10.4 | 31.12 | 2.63 | 26.33 | 3.52 |

### 1C. Compound I/Compound II Microparticle Coating

A 10 mg portion of colloidal gold-nucleated Fab particles, as prepared in 1A was placed in a centrifuge filter. To the particles, 200 μL of a solution containing 2 mg/mL Compound I (PEI-APTAC-EITC) in methanol was added and incubated for 15 minutes at room temperature. The solutions became colorless and the particles were visibly coated with Compound I. Any excess Compound I (PEI-APTAC-EITC) was removed by spinning the particles in PTFE filters (0.2 μm (Amicon, Ultrafree-CL)) at 3000 rpm for 3 minutes. Particles were then rinsed using CHCl₃ and spinning again at 3000 rpm for 3 minutes.

Solvent was further removed from the Compound I-coated polypeptide microparticles by drying them in a vacuum oven. Coating solutions were made, using Compound II (MD-methacrylate) and polyethylene glycol (PEG, 30%), wherein Compound II was present at concentrations of 500 μg/mL or 1 mg/mL. The Compound H coating solutions were added to the particles coated with Compound I (PEI-APTAC-EITC). Particles were mixed thoroughly in suspension. Added to the suspension was 10 uL of trolamine and the mixture was placed under a UV light for 60 seconds using Blue Wave illuminator (Dymax Blue-Wave™ 200 operating at 330 nm between about 1 and 2 mW/cm²). The red color of coating turned faint yellow.

Particles were then spun down and excess coating solution was decanted. Samples were replenished with 1 mL of PBS and assayed for protein release utilizing the ELISA Assay and Spectrophotometric Protein Determination.

The method provided a coated polypeptide microparticles with a Fab core, a Compound I coated layer on the Fab core (initiator), and a Compound II coated layer on the Compound I coated layer (crosslinked).

### 1D. Compound V/Compound II Microparticle Coating

Coating solutions for the prepared colloidal gold-Fab microparticles, as prepared in Example 1A were prepared as follows. Generally, Compound V (TEMED-DQ) was found to be not readily soluble in chloroform, methanol, or DDW at pH 7. Thus, Compound V (10 mg) was dissolved in solvent containing 100 μL of methanol and 900 μL of chloroform. A solution of 100 μL of the 1:9 MeOH.CHCl₃ with Compound V was added to 5 mg of Fab particles (prepared in Example 1A). The mixture was allowed to react at room temperature for 30 minutes.

The Compound V-coated Fab microparticles were then dried in the vacuum oven until solvent was evaporated. A second coating solution was prepared dissolving Compound II (MD-methacrylate) in a 30% w/v PEG 20 kDa solution in DDW at pH 7, at concentrations of 500 μg/mL, 1 mg/mL, or 50 mg/mL. The compound II/PEG solutions, in a volume of 1 mL, were added to the Compound V-coated Fab particles. Particles were mixed thoroughly and then placed under the UV lamp for 60 seconds using Blue Wave illuminator (Dymax Blue-Wave™ 200 operating at 330 nm between about 1 and 2 mW/cm²).

After illumination, particles were spun down and excess MD-methacrylate/PEG solution was decanted. For elution studies, samples were replenished with 1 mL of PBS in microcentrifuge tubes. At specific time intervals, the particles were spun down and the PBS was removed for analysis utilizing the ELISA Assay and Spectrophotometric Protein Determination. Particles were then resuspended in fresh PBS.

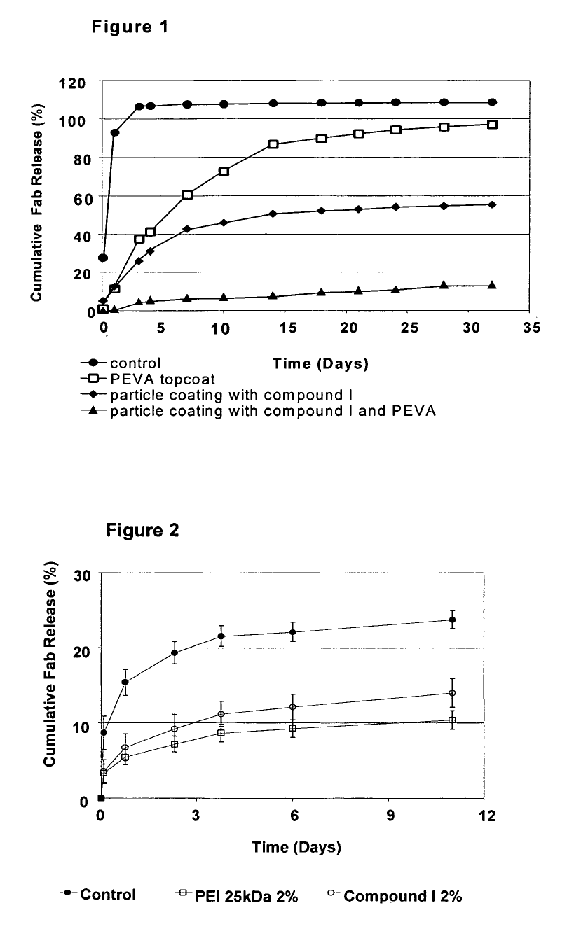

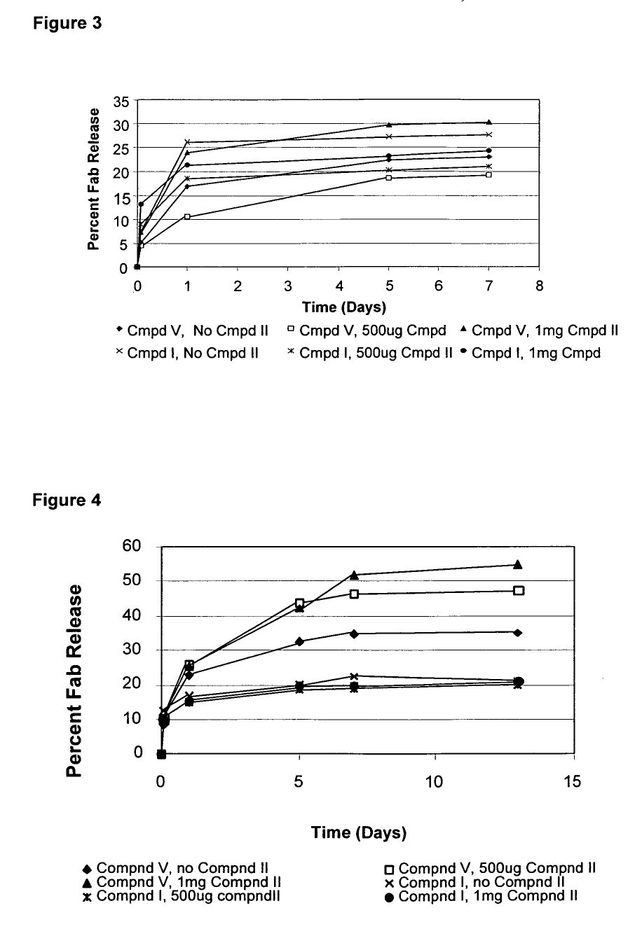

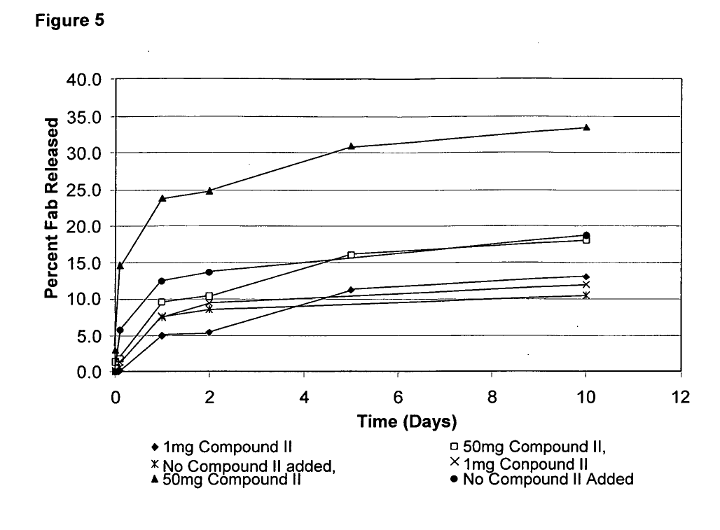

Results are illustrated in FIGS. 3, 4, and 5, in which time (days) is represented on the X-axis, and percent release (%) of the Fab from the microparticles is represented on the Y-axis.

Results indicated that particles coated with Compound V and crosslinked Compound II (1 mg) gave a 10% release of Fab every 2 to 3 days.

Without intending to be bound by a particular theory, the coating of Compound I (PEI-APTAC-EITC) on the microparticles may be described as an initiator layer that promotes formation of a polymerized layer from the maltodextrin macromers via free radical polymerization initiation.

As illustrated in Graph 3, results indicate that increased recovery of protein can be related to the increased amount of Compound II (methacrylated polysaccharide) used in the crosslinking. Generally, it was observed that the higher concentration of methacrylated polysaccharide increased the likelihood that the initiator would react with the polysaccharide, and thus slowed the release of Fab from the microparticles.

### EXAMPLE 1D(2)

Coating solutions for the prepared colloidal gold-Fab microparticles as prepared in Example 1A were prepared as follows. Compound V (TEMED-DQ, 10 mg) was dissolved in solvent containing 100 μL of methanol and 900 μL of chloroform. 100 μL of a 10 mg/mL solution of Compound V in 1:9 MeOH.CHCl₃ was added to 50 mg of Fab particles (prepared in Example 1A). The mixture was allowed to react at room temperature for 30 minutes.

The Compound V-coated Fab microparticles were then dried in the vacuum oven until solvent was evaporated. A second coating solution was prepared dissolving Compound II (MD-methacrylate) a concentration of 50 mg/mL in a 30% w/v PEG 20 kDa solution in DDW at pH 7. Compound II/PEG solution, in a volume of 1 mL, was added to the Compound V-coated particles. Particles were mixed thoroughly and then placed under the UV lamp for 60 seconds using Blue Wave illuminator (Dymax Blue-Wave™200 operating at 330 nm between about 1 and 2 mW/cm²). After mixing thoroughly the suspension was irradiated again for 60 seconds. The suspension was lyophilized using a bench-top lyophilizer. Following lyophilization, PEG was extracted using chloroform. Once no soft spots were observed, the dry cake was transferred to a 50 mL centrifuge tube. A 20 mL aliquot of chloroform was added. The PEG dissolved, rendering a cloudy fine protein suspension. The chloroform was dispensed in 4 PTFE filters 0.2 μm (Amicon, Ultrafree-CL™) and centrifuged at 5500 rpm, 10° C. for 15 minutes. Using glass pipettes, fresh chloroform was added. This washing procedure was done 3 times in total. 97 mg of solids was recovered after lyophilization and removal of PEG.

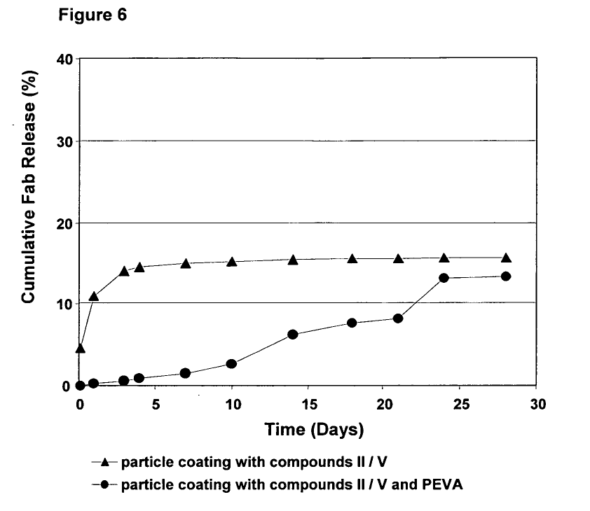

For the preparation of a device coating solution, the particles were resuspended in chloroform adding a solution containing 20 mg/mL pBMA, 20 mg/mL pEVA and 11 mg/mL 1000PEG₄₅PBT₅₅. Particles were mixed with coating solution at 60% w/w and 30% w/w concentrations. Eight intravitreal implants were coated per formulation. Four intravitreal implants were additionally topcoated with a 20 mg/mL pEVA solution using ultrasonic spray as described herein. After drying over night in a nitrogen box at room temperature the coated intravitreal implants were placed in 1 ml PBS for Fab release assays. At specific time intervals the elution medium was removed and analyzed. The elution medium was assayed for Fab release, utilizing the ELISA Assay. The values in the table are time (days) versus cumulative release (%) as calculated from theoretical total loading. Results of Fab release are shown in Table 6 and FIG. 6.

TABLE 6

| | | particle coating with |
|---|---|---|
| time | particle coating with | compounds II/V and |
| (days) | compounds II/V | PEVA topcoat |
| 0.1 | 4.56 | 0.05 |
| 1 | 10.96 | 0.18 |
| 3 | 14.06 | 0.56 |
| 4 | 14.52 | 0.89 |
| 7 | 14.99 | 1.52 |
| 10 | 15.24 | 2.60 |
| 14 | 15.46 | 6.28 |
| 18 | 15.54 | 7.59 |
| 21 | 15.60 | 8.13 |
| 24 | 15.66 | 13.17 |
| 28 | 15.71 | 13.43 |

### EXAMPLE 2

### Formation of Fab Microparticles with Amphiphilic Polymer Microparticle Coating

Microparticles were coated with Compound I as described in Example 1A above (4 mg colloidal gold-Fab microparticles with 0.2 mg Compound I). The coated particles were dried as described in Example 1A. The coated colloidal gold-Fab microparticles were resuspended in 1 ml of chloroform in a microcentrifuge tube.

Compound VIII (poly(ethylene glycol)-di(imidazolyl carbonate) PEG-CDI), MW1000Da, prepared as described in commonly assigned U.S. Pub. No. 2008/0039931, was added to the particles in the following ways:

| | |
|---|---|
| Samples1-3. | An aliquot of Compound VIII (PEG-CDI) (100 μL) was dissolved in 500 ml |
| | chloroform. The resulting PEG-CDI/chloroform solution was added to the |
| | particles, in amounts of 30 μL, 100 μL, or 230 μL, and the particles were |
| | maintained at room temperature and monitored for dissolution in water |
| | regularly. |
| Sample 4. | Dry Compound I-coated colloidal gold-Fab particles were resuspended in pure |
| | Compound VIII (PEG-CDI) (200 μL). |
| Sample 5a. | Alternatively Fab-microparticles were coated with Compound I and |
| | subsequently with Compound VIII in a one-pot reaction without removing the |
| | 30% w/v PEG that was present at the formation of the Fab particles after |
| | lyophilization. |
| Sample 5b. | Compound I (APTAC-EITC-PEI) (0.2 mg) was added to the suspension of |
| | colloidal gold-Fab microparticles (4 mg) in chloroform where PEG 30% w/v |
| | was still present. After the particles were coated by Compound I and the |
| | solution had become colorless, Compound VIII (200 μL) in 1 mL of |
| | chloroform was added. |

The resulting coated particles (Samples 1-5b) were dried in a vacuum oven. Particles were found insoluble when suspended in PBS. The particles were suspended in 1 ml regular PBS (0.01 M) in microcentrifuge tubes. At specific time intervals, the particles were spun down at 5000 rpm for 5 minutes. The elution medium was removed and analyzed, and the particles were resuspended in fresh PBS. Controlled release was measured with ELISA Assay and Spectrophotometric Protein Determination. Results are summarized in Table 7.

TABLE 7
Release of Fab-fragment from coated particles (in μg)

| Time | | | | | | |
|---|---|---|---|---|---|---|
| (hrs) | Sample 1 | 2 | 3 | 4 | 5a | 5b |
| 1 | 2489.60 | 3248.72 | 2681.76 | 1944.64 | 3328.00 | 2649.28 |
| 4 | 10.38 | 5.23 | 13.93 | 1.80 | 5.06 | 5.76 |
| 24 | 11.11 | 15.11 | 16.31 | 10.29 | 1.48 | 1.01 |
| Total | 2511.09 | 3269.06 | 2712.00 | 1956.73 | 3334.54 | 2656.05 |

Results indicated that not all the protein was retrieved. A burst was noticed where most of the loaded protein was released.

### EXAMPLE 3

### Formation of Fab Microparticles with Polyacrylamide Matrix Coating

Generally, NOS(N-oxysuccinimide) groups are very reactive with free amines. Compound IV is polydimethylacrylamide polymer with pendent NOS groups and BBA photoreactive groups. Compound IV was soluble in water or DMSO, and was freely soluble in chloroform.

Fab particles (prepared as described in Example 1A) were coated with Compound I (PEI-APTAC-EITC, 0.05 mg/mL Fab) as described in Example 1B, without presence of the PEG 30% w/v. A solution of Compound IV (BBA-DMA-NOS) in chloroform (10 mg/mL) was made fresh before every experiment. Compound IV was added to the Compound I-coated Fab particle suspension in chloroform (330 μg Compound IV to 5.7 mg of Fab).

After reacting at room temp for 1 hour, a sample was taken. The chloroform was dried and the sample was suspended in DDW and a microscopic image was taken at a magnification of 500× using polarized light. Particles coated with Compound I and Compound IV were placed in PBS for controlled release assay, in accordance with the ELISA Assay. Results are shown in Table 8 below showing cumulative release of Fab from the particles based on theoretical loading.

TABLE 8

| time (hours) | no PEG | w/ PEG |
|---|---|---|
| 2 | 61% | 91.3% |
| 26 | 3.5% | 3.3% |

In this example, the coating processes using PEG (20 kDa) in solution, appeared to results in a less stable particle coating (shell) on the Fab core, as indicated by the faster release of Fab from these microparticles.

### EXAMPLE 4

### Formation of BSA Microparticles with Polysaccharide Coating

Microparticles containing BSA were prepared as follows. BSA (fraction V, ICN, Aurora, Ohio) was dissolved at 20 mg/mL in DDW. To 20 mL of the BSA solution, 4 g poly(vinylpyrrolidone) (PVP) (Kollidon 90, BASF) was added. The mixture was frozen at −20° C. and lyophilized using a vacuum oven at room temperature. The PVP was then extracted with chloroform by adding the lyophilized powder to 20 mL chloroform in a 50 mL centrifuge tube, and the resultant BSA particles were dried and stored as a dry powder until use.

A solution of an acrylated polyalidtol in DMSO was prepared by dissolving 100 mg of Compound VII (acrylated polyalditol), in 400 μL of DMSO. The following polysaccharides were utilized, wherein degree of substitution is (DS) indicated in parenthesis for each compound:

Compound VII, DS (0.75)

Compound VII, DS (0.25)

BSA particles in an amount of 10 mg were added to a tared Eppendorf tube. Appropriate amounts of the Compound VII solution were added to each Eppendorf tube at a ratios 5:1, 1:1 and 1:3 (BSA to Compound VII). DMSO was added where needed to keep the total volume at either 50 μL or 120 μL total.

Formulations for the microparticles are summarized in Table 9 below.

TABLE 9
Quantities and Volumes used in Formulations

| | | | CmpdVII/250 | Additional | Total |
|---|---|---|---|---|---|
| | BSA | Cmpd VII | mg/ml DMSO | DMSO added | DMSO |
| sample | (mg) | (mg) | solution (μl) | (μl) | (μl) |
| 1 | 9.8 | 2 | 8 | 42 | 50 |
| 2 | 10.1 | 10 | 40 | 10 | 50 |
| 3 | 10 | 30 | 120 | 0 | 120 |
| 4 | 10.1 | 2 | 8 | 112 | 120 |

Each solution was sonicated on setting 1.5, for 10 seconds, using the pulsing mode (0.5 sec on/0.5 sec off). After sonication, 5 mg of DBDS (Compound VI), dissolved in 50 μL of DI water was added to 10 mL of PEG solution (what PEG was used?). The PEG/Compound VI solution was vortexed vigorously for 30 seconds. The final concentration of Compound VI in the solution was 0.5 mg/mL.

After vortexing, 1 mL of the 30% PEG/Compound VI solution was added to the BSA/Compound VII/DMSO solution at room temp and vortexed vigorously. The mixture was then placed under a UV Lamp for 60 seconds (Dymax Blue-Wave™ 200 operating at 330 nm between about 1 and 2 mW/cm²). Following UV cross-linking, the sample was spun down at 5000 rpm for 10 minutes. The PEG was decanted, and the resulting wet particles were frozen and lyophilized.

Chloroform was added (approximately 1 mL) to the lyophilized particles. The solution was spun down at 5000 rpm for 8 minutes, and the chloroform was decanted from the solid particles. Another 1 mL of chloroform was added to the Eppendorf tube, and the solution was transferred to a centrifuge filter. The solution was spun down at 3000 rpm for 3 minutes. This process was repeated 3 more times, each time enough chloroform was added to fill the filter tube. After the final spin, samples were taken for microscopic inspection. Excess solvent was removed using a vacuum oven.

For controlled release experiments, 1 mL of DI water was added to microparticles. After a period of BSA release from the microparticles, the suspension was spun down at 5000 rpm for 10 minutes. The aqueous phase was drawn off and analyzed for BSA quantification. Fresh DI water (1 mL) was added to the microparticles. The microparticle suspension was sonicated briefly using a probe sonicator on setting 1.5, for 10 seconds, using the pulsing mode (0.5 sec on/0.5 sec off). The microparticle suspension was spun down at 5000 rpm for 10 minutes. The aqueous phase was drawn off and analyzed for protein quantification.

In addition, the particles were viewed by light and electron microscopy, as well as analyzed by RAMAN spectroscopy (results not shown). These analysis techniques Results demonstrate that a core-shell pattern was found. Images from the RAMAN spectroscopy revealed that the maltodextrin coating encompassed the protein particles almost completely, confirming a core-shell formation.

Protein quantification was performed using the Bradford reagent (Sigma). Samples (100 μL of protein solution) were placed in a 96-well plate and the Bradford reagent, 100 μL, was added to each sample. Samples were read at 595 nm. Results are shown in Table 10.

Elution conditions were as follows: Particles were weighed and put in DDW in an eppendorf tube tube (1 ml) and left for 2 hours and then centrifuges Supernatant was analysed spectrophotometrically at A280. DDW added and sonicated with probe sonicator, centrifuged again, and then the supernatant analyzed.

TABLE 10

| | | | Total DMSO | release (μg) | release (μg) |
|---|---|---|---|---|---|
| Sample | Cmpd VII | DS | (μL) | in DI water | after sonication |
| 1 | 2 mg | 0.25 | 50 | 14365 | 556 |
| 2 | 10 mg | 0.25 | 50 | 10852 | 1050 |
| 3 | 30 mg | 0.25 | 120 | 8961 | 814 |
| 4 | 2 mg | 0.25 | 120 | 8291 | 730 |
| 5 | 2 mg | 0.75 | 50 | 9599 | 760 |
| 6 | 10 mg | 0.75 | 50 | 8703 | 5008 |
| 7 | 30 mg | 0.75 | 120 | 1000* | 6918 |
| 8 | 2 mg | 0.75 | 120 | 6154 | 735 |

*Below detection, but estimated to be approx 1000ug

Results indicated that the optimal formulation for this experiment was sample #7 using acrylated polyalditol (Compound VII) with a degree of acrylate substitution of 0.75. Results indicated that only about 10% of the protein eluted from the microparticle after the first addition of water, while after sonication formulation #7 lost about 70% of its protein.

This Example was also performed utilizing Fab-particles made with colloidal gold, which were coated with the acrylated polyalditol (Compound VII) like the BSA particles. Similar elution results were observed.

### EXAMPLE 5

### Formation of Microparticles Via Redox

In this Example, non-specific Fab was tagged with fluorescamine and made into particles with Compound II using a redox method. The fluorescamine-tagged Fab was used to determine particle integrity and protein loss.

The following polysaccharides were utilized, wherein degree of substitution is (DS) indicated in parenthesis for each compound:

Compound II, DS (0.45)

Compound II, DS (0.1)

Compound II, DS (0.25)

The following solutions were prepared:
(1) Acetone, 1 mL, was combined with 20 mg of fluorescamine in an amber vial to make a 20 mg/mL Fluorescamine solution.
(2) Phosphate buffer: 25 mM, pH 7.1: combination of aqueous solutions 117 mL of 24 g/L monobasic with 183 ml 28.4 g/L dibasic sodium phosphate in DDW.
(3) Tetramethylethylenediamine (TEMED, Sigma): TEMED (3 mL) was combined with phosphate buffer (6 mL, 25 mM, pH 7.1) and 4N HCl (6 mL)
(4) Sodium persulphate (NaPS): 886 mg dissolved in 20 mL DDW

A 12.5 μL aliquot of flourescamine (20 mg/mL in acetone) (TCI America) was added to 4 mL of nonspecific Fab (15.4 mg/mL, Southern Biotech). The product was run through a desalting column (Econo-Pac 10 DG, Bio-Rad) at 2 mL per column to eliminate any fluorescamine that did not react with Fab. Fluorescamine-labeled Fab was eluted by adding 4 mL of PBS to the columns. Eluent was collected and placed into 2 centrifuge filter tubes (10 kDa cutoff) (Microsep 10 k Omega, Pall). The protein was spun down for 45 minutes at 5500 rpm.

Concentrated fluorescamine-Fab (400 μL, approximately 25 mg/mL, 10 mg) was added to a 15 mL centrifuge tube with 60 mg of Compound II (MD-methacrylate) with different degrees of substitution. An amount of 5 mmol PBS (100 μL) was added to each centrifuge tube. The solution was mixed until the Compound II was dissolved. While vortexing vigorously, 3 mL of the PEG 20 kDa, 30% w/w was added to the fluorescamine-Fab/Compound II mixture (the total vortex time was 30 seconds).

To initiate polymerization, 100 μL of TEMED was then added, and the solution was vortexed for 1 minute. Next, 180 μL of NaPS was added to the mixture and tumbled 3 times. The mixture was placed in the freezer −20° C. for 1 hour, and then on dry ice for another 15 minutes. The mixture was lyophilized by placing in the vacuum oven overnight at room temperature.

Acetone, 8 mL, was added to the dried cake of microparticles. The solution was spun down at 5000 rpm for 10 minutes. Not all of the cake dissolved in acetone, so the solvent was decanted. Chloroform, 8 mL, was added until all of the cake went into solution. The solution was spun down at 5000 rpm for 10 minutes. Solvent was decanted and another 8 mL of chloroform was added. This extraction step was repeated 3 times in total.

The remaining particles were dried in the vacuum oven for 1 hour. Following this the and assay was performed to assess Fab release by resuspending the microparticles (how many) in 5 mL of DI water at 37° C. for about 4 hours. The solution was spun down at 500 rpm for 8 minutes. The water phase was collected for analysis and then another 5 mL of DI water was added to the particle solution. The solution was sonicated for 10 seconds with an ultrasonic probe on setting 2. The water phase was collected for analysis again. Another 1 mL of DI water was added to the microparticles, and the solution was sonicated for 15 seconds on setting 3. The sample was saved for analysis.

For this procedure, since the fluorescamine is sensitive to light, samples were wrapped in tin foil during drying and storage steps.

Results: Under the florescent microscope, there was evidence of particle production for all of the acrylated maltodextrins used to coat the Fab microparticles. The Compound II (DS 0.25) seemed to form the highest quality particles, which were separated and spherical in nature as viewed microscopically, as well as producing the largest quantities of particles. For the Compound II (DS 0.1), particles were formed, but in smaller quantities relative to the Compound II (DS 2.5), while the Compound II (DS 0.45) hardly formed any particles at all. Results of Fab release are shown in Table 11.

TABLE 11
Release of fluorescamine-Fab (mg)

| | In Water | Sonication | |
|---|---|---|---|
| Sample | Phase | 1 | Sonication 2 |
| Compound II (0.45) | 0.29 | 0.05 | 1.21 |
| Compound II (0.10) | 0.84 | 0.00 | 0.17 |
| Compound II (2.5) | 0.20 | 0.40 | 4.80 |

From the Fluorescamine results, the only significant protein losses were due to severe sonication which was thought to destroy the structure of the coated particles. The relatively low loss of protein due to the addition of water and the visual evidence of protein particles under microscope indicated formation of a cross-linked maltodextrin shell around the fluorescamine-Fab core and modulation of Fab release using this shell.

The elution profiles indicated that there was very little loss of Fab after the addition of water. It was only after a highly penetrating sonication, that protein elution from the particles was observed.

### EXAMPLE 6

### Incorporation and Release of Protein-Containing Microparticles from Polymeric Coating

An aqueous IgG solution was prepared consisting of 10% specific rabbit-α-goat and 90% non-specific protein (Lampire) at 20 mg/mL in solution in Phosphate buffer (no NaCl).

Acrylated maltodextrin (Compound III) was dissolved in the IgG solution at a 1:2 IgG:MD-Acrylate w/w ratio. Particles were obtained by slowly mixing in a 30% w/v PEG 20 kDa solution with 0.5 mg/mL Compound VI (DBDS) while vortexing the IgG:MD-Acrylate solution (1 mL). By adding DBDS to the PEG-phase, the formed particles could be crosslinked. The crosslinked particles were formed by a 5 minute-UV irradiation. UV irradiation was done in the cold room using Dymax lamp at 4° C. while stirring the PEG-particle suspension on ice. Resultant particles were isolated by centrifugation at 5,000 rpm for 10 minutes. Remaining PEG was further removed by adding 5 mL isopropyl alcohol (IPA) to the residue. The suspension was vortexed and spun at same settings. The washing with IPA was repeated. Subsequent washing was done with 5 mL chloroform.

A weighed amount of the IgG/MD-Acrylate particles (10 mg) was incubated in 1 mL of PBS to characterize the release kinetics. At predetermined intervals, the eluent was removed from the microcentrifuge tube, and 1 mL of fresh eluent solution (1×PBS) was added to the microcentrifuge tube containing the particles. The eluent samples in 96 well plates were analyzed for activity of the IgG using the ELISA Assay.

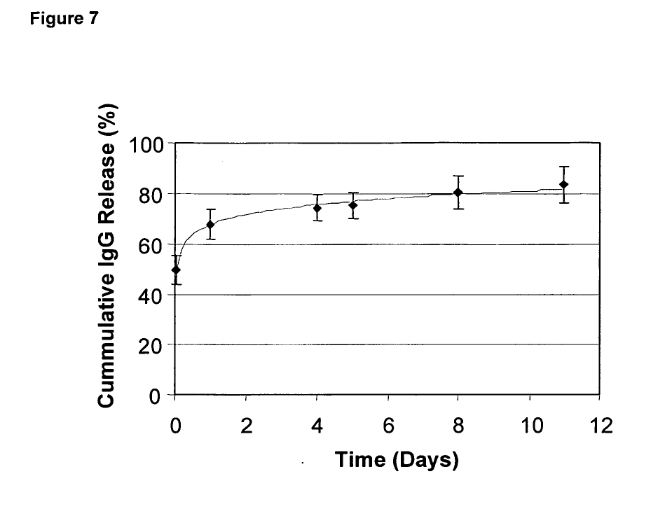

As illustrated in FIG. 7, a burst of IgG was seen of around 50% in the first hour. Total protein was measured using the Bradford reagent (Sigma). The burst was caused by particles that consist of mostly of either IgG alone or by particles with uncompleted crosslinking. Using the ELISA assay, a total release of about 85% (active IgG w/w total active IgG) was measured over 11 days, and the particles were still releasing functional IgG protein.

### Polymeric Coating Composition.

IgG/MD-Acrylate particles described above were loaded into a pBMA/pEVA/PEG₁₀₀₀-45PBT-55 coating solution at 30% w/w IgG/MD-acrylate microparticles. A polymeric coating composition was prepared using the components and amounts thereof as indicated in Table 8. In a 15 mL chloroform suspension of IgG/Compound III microparticles (1:2 ratio at 0.83 mg/ml), 6.3 mg of PEG₁₀₀₀-45PBT-55, 12.5 mg pEVA, and 12.5 mg pBMA were dissolved while shaking the mixture for 30 minutes on an orbital shaker at 37° C. Four helical intravitreal implants were coated.

The total loading of IgG on each substrate was approximately 50 μg. (150 μg of IgG/MD particles in 500 μg coating). Results indicated that 10% of the IgG was active (approximately 5 μg). An additional topcoat with pEVA/pBMA at a 1:1 ratio was applied to implant numbers 9 and 10. See Table 12 for coating weights.

TABLE 12
Coating weights

| | | | | | | pEVA/pBMA | Total | Total | Active |
|---|---|---|---|---|---|---|---|---|---|
| Implant | IgG | Cmpd | 1000PEG₄₅ | pBMA | pEVA | top | Coating | IgG in | IgG |
| nr. | (%) | III (%) | PBT₅₅ (%) | (%) | (%) | coat (mg) | wt (μg) | (μg) | (μg) |
| 7 | 9.5 | 19 | 14.6 | 28.6 | 28.6 | | 485 | 46.07 | 4.607 |
| 8 | 9.5 | 19 | 14.6 | 28.6 | 28.6 | | 500 | 47.5 | 4.75 |
| 9 | 9.5 | 19 | 14.6 | 28.6 | 28.6 | 0.228 | 458 | 43.51 | 4.35 |
| 10 | 9.5 | 19 | 14.6 | 28.6 | 28.6 | 0.259 | 474 | 45.03 | 4.50 |

Prior to coating, the matrix particle matrix suspension was very fine and extremely stable. The obtained coatings were smooth under visual inspection. Total loading of IgG was approximately 50 μg. (150 μg IgG/MD particle in 500 μg coating). The final coating weight was approximately 500 μg.

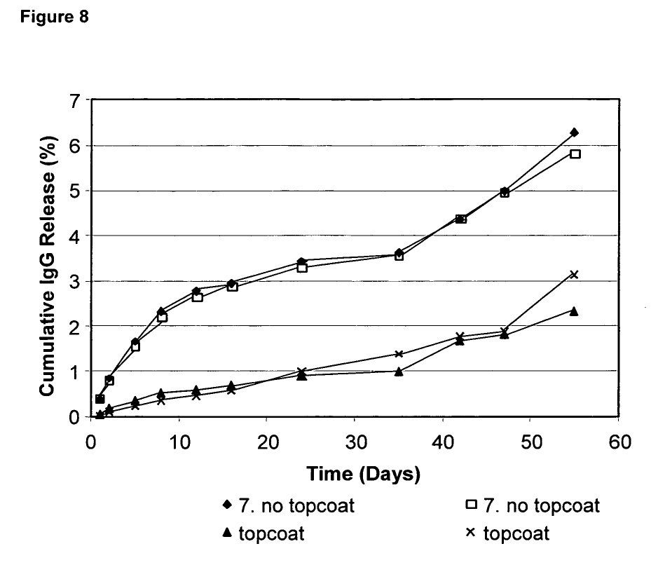

FIG. 8 shows the results for the controlled release of IgG from the IgG/MD-acrylate particles in the pBMA/pEVA/PEG₁₀₀₀-45PBT-55 coated matrix from four implants. The addition of a pBMA/pEVA topcoat, provides additional control of the release of IgG. In FIG. 8, time (days) is represented on the X-axis, and cumulative release (%) is represented on the Y-axis.

Active IgG was measured by ELISA. Table 13 shows the controlled release of active IgG with and without topcoats up to 55 days. The numbers in the table represent cumulative release IgG (%) by calculated by total theoretical loading.

TABLE 13

| | |
|---|---|
| Time | Sample Number |

| (days) | 7 | 8 | 9 | 10 |
|---|---|---|---|---|
| 1 | 0.39 | 0.38 | 0.06 | 0.03 |
| 2 | 0.86 | 0.78 | 0.19 | 0.09 |
| 5 | 1.67 | 1.54 | 0.35 | 0.24 |
| 8 | 2.32 | 2.20 | 0.51 | 0.36 |
| 12 | 2.77 | 2.64 | 0.61 | 0.47 |
| 16 | 2.95 | 2.87 | 0.67 | 0.57 |
| 24 | 3.43 | 3.29 | 0.91 | 1.02 |
| 35 | 3.63 | 3.56 | 1.01 | 1.39 |
| 42 | 4.36 | 4.39 | 1.66 | 1.77 |
| 47 | 4.99 | 4.96 | 1.82 | 1.88 |
| 55 | 6.26 | 5.80 | 2.31 | 3.14 |

### EXAMPLE 7

### Formation of Polysaccharide Microparticles Containing Fab

Microparticles containing Fab were prepared as follows. Non-specific Fab (Lampire) and acrylated maltodextrin (Compound III) were combined at 2:1, 1:1, 1:2 and 1:4 protein:maltodextrin ratios. Fab microparticles were obtained by slowly mixing in a 30% w/v PEG 20 kDa solution with 0.5 mg/mL Compound VI (DBDS) while vortexing the Fab/Compound III solution at room temperature, with relatively controlled rapid addition. By adding Compound VI to the PEG-phase, the formed particles could be crosslinked. This was achieved by a 0.5 or 3 minute-UV irradiation at 4° C. and stirring the PEG-particle suspension on ice. The particles were isolated by centrifugation. PEG was further removed by subsequent washing steps with isopropyl alcohol (IPA) and finally chloroform.

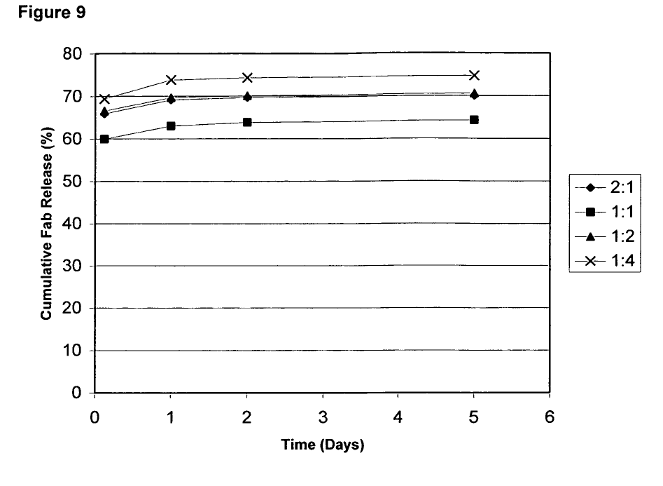

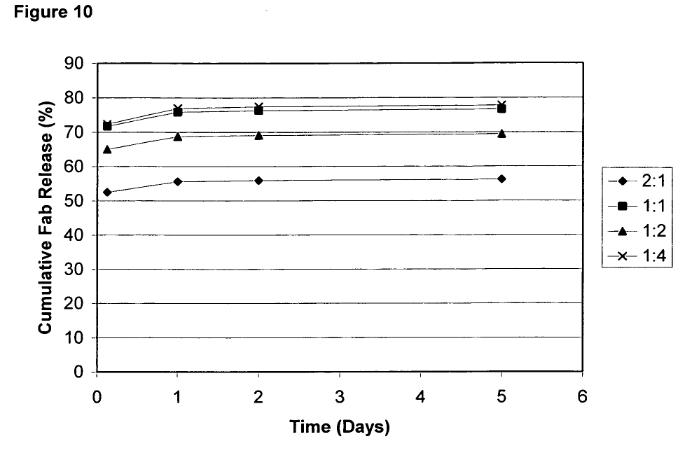

A weighed amount of particles was incubated in 1 mL of PBS to characterize the release kinetics. Other factors investigated were Fab:maltodextrin ratio and UV irradiation time. For particle formation, best results were obtained by adding the Fab/maltodextrin to the 30% PEG. Results are illustrated in FIGS. 9 and 10, which compared 0.5 minutes (FIG. 10) with 3 minutes (FIG. 9) for irradiation to crosslink the polymeric matrix.

The release rate of Fab from the microparticles was assayed using the ELISA Assay and Spectrophotometric Protein Determination. Results demonstrated a burst of Fab upon resuspension of the microparticles in the release medium, similar to IgG containing particles.

After 5 days, the buffer was removed and the particles were incubated with amylase containing PBS (10 mg/L) for 24 hours. Maltodextrin particles that did not contain Fab were included as a control. Results of the incubation with amylase demonstrated that no additional Fab was released from the microparticles. No background signal was observed for the control MD particles that lacked Fab.

### EXAMPLE 8

### Formation of Crosslinked Fab Microparticles

For this Example, a solution of Fab (Southern Biotech) at 20 mg/mL in 5 mM phosphate buffer, pH 7 was prepared.

Colloidal gold (VWR, 5 nm, 0.01% w/v) (VWR, cat#IC15401005) in a volume of 5 μl was placed in microcentrifuge tube. An amount of the Fab protein solution was added to the colloidal gold to provide an amount of Fab of 3.6 mg or 2 mg. Methacrylated maltodextrin (Compound II, Example 3) at a concentration of 10% w/w or 50% w/w (0.4 or 2 mg) was added to the Fab/colloidal gold solution.

The colloidal gold/Fab/maltodextrin solutions were heated to 50° C. A PEG solution (20 kDa PEG dissolved in 30% w/v water, at pH 5) was warmed to 50° C., and 70 μl of the PEG solution was added to the colloidal gold/Fab/maltodextrin solutions.

The PEG/colloidal gold/Fab/maltodextrin solutions were then cooled at −20° C. for 60 minutes. The frozen mixtures were subsequently lyophilized at room temperature (benchtop lyophilizer, no temperature or vacuum pressure control). PEG was extracted using chloroform. For chloroform extract, 1 mL of chloroform was added to the microtube with lyophilized Fab microparticles, and the Fab particles creamed on the surface of the solution. The particles were aspirated with a glass pipette and mixed in 1 mL of fresh chloroform. The extraction/aspiration was repeated three times. A sample of the Fab microparticles in chloroform was dried on a microscope glass slide and analyzed using RAMAN imaging. RAMAN imaging revealed asymmetric crystallization where the core of the particles was formed of Fab and the outer layer of the particles consisted of the maltodextrin.

The crosslinking can be accomplished by adding Compound I (PEI-APTAC-EITC) or other photocrosslinkable initiator to a 30% w/v PEG 20 kDa solution in DDW. The solution is cooled to 4° C. and the particles are suspended in the solution. While on ice the mixture is placed under a UV source for 60 seconds.

In other aspects, thermosensitive initiators can be used to crosslink the formed microparticles. The thermosensitive initiator 2,2′-azobis(2,4-dimethylvaleronitrile) is commercially available from DuPont, Wilmington, Del., under the trade designation Vazo™ 52. For crosslinking, the Fab-maltodextrin microparticles are contained in a PEG 20 kDa cake. A solution of water soluble Vazo™ 52 is added in PEG₄₀₀ at 10 mg/mL.

The Fab/maltodextrin in PEG-cake is thoroughly vortexed in the PEG₄₀₀ solution and placed at 50° C. in the oven. PEG 20 kDa melts at this temperature and dissolves in the PEG₄₀₀ solution. The Vazo™ 52 initiator slowly decomposes and crosslinks the particles over a 60-minute period.

What is claimed is:

1. A microparticle comprising:
(a) a core comprising predominantly polypeptide; and
(b) a microparticle coating, the microparticle coating comprising a crosslinked polymeric matrix, wherein the polypeptide is capable of being released from the microparticle and wherein the microparticle coating is able to modulate release of the polypeptide from the microparticle.

2. The microparticle of claim 1 wherein the polypeptide comprises a Fab or Fab′2 fragment.

3. The microparticle of claim 1 wherein the polypeptide is present in an amount of 50% wt or greater in the core.

4. The microparticle of claim 3 wherein the polypeptide is present in an amount of 70% wt or greater in the core.

5. The microparticle of claim 1 which comprises polymerized groups that covalently couple polymer together forming the crosslinked polymeric matrix.

6. The microparticle of claim 1 wherein the crosslinked polymeric matrix comprises a biodegradable polysaccharide selected from the group consisting of maltodextrin, polyalditol, and amylose.

7. The microparticle of claim 1 wherein the crosslinked polymeric matrix comprises a polymer having a molecular weight in the range of 1,000 Da to 100,000 Da.

8. The microparticle of claim 1 wherein the weight ratio of the core to the microparticle coating is in the range of 96:4 to 50:50.

9. The microparticle of claim 1 wherein the microparticle coating comprises a polymerization initiator proximal to the core.

10. A method for forming a microparticle comprising a core comprising
predominantly polypeptide and a microparticle coating comprising a crosslinked polymeric matrix, the method comprising the steps of:
(a) in a liquid composition, providing a core particle comprising predominantly polypeptide;
(b) mixing the core particle with a first component comprising a first reactive group;
(c) mixing the core particle with a second component comprising a polymer and a pendent a second reactive group; wherein either:
(i) the first reactive group is reactive with the second reactive group, thereby forming the crosslinked polymeric matrix, or
(ii) the first reactive group comprises a polymerization initiator group and the second reactive group comprises a polymerizable group, and the method additionally comprises (d) activating the initiator group to cause polymerization of the first component, thereby forming the crosslinked polymeric matrix, and
wherein step (b) can be performed before, after, or at the same time as step (c).

11. The method of claim 10 where, in step (a), the core particle is present in the composition at a concentration in the range of 4 mg/mL to 50 mg/mL.

12. The method of claim 10 where, in step (b), the second component is mixed with the core particle at a weight ratio in the range of 2:1 to 0.05:1.

13. The method of claim 10 where, in step (c), the first component is mixed with the core particle at a weight ratio in the range of 0.5:100 to 10:100.

14. The method of claim 10 where, wherein the first component comprises a water soluble polymerization initiator having a molecular weight of about 500 or less.

15. The method of claim 10 comprising a step of adding a phase separation agent to the liquid composition at concentration in the range of 100 mg/mL to 500 mg/mL, wherein the phase separation agent comprises an amphiphilic compound.

16. The method of claim 15 where the step of adding the phase separation agent is performed at a temperature in the range of 20° C. to 55° C.

17. A method for forming a microparticle comprising a core comprising
predominantly polypeptide and a microparticle coating comprising a crosslinked polymeric matrix, the method comprising the steps of:
providing a liquid composition comprising polypeptide, nucleating agent, and polymer comprising pendent reactive groups;
(b) heating the composition to a temperature above room temperature;
(c) adding a phase separation agent comprising an amphiphilic compound to the composition;
(d) cooling the composition formed in step (c);
(e) extracting at least a portion of the phase separation agent; and
(f) activating the pendent reactive groups to crosslink the polymer to form the crosslinked polymeric matrix.

18. The method of claim 17 wherein the polypeptide is present in the composition in step (a) at a concentration in the range of 10 mg/mL to 50 mg/mL

19. The method of claim 17 wherein the nucleating agent is present in the composition in step (a) at a concentration in the range of 1 μg/mL to 10 μg/mL.

20. The method of claim 17 wherein the polymer comprising pendent reactive groups is present in the composition at a concentration in the range of 1 mg/mL to 30 mg/mL.

21. The method of claim 17 where the composition is heated to a temperature in the range of 30° C. to 70° C. in step (b).

22. The method of claim 17 where the phase separation agent present in the composition at a concentration in the range of 100 mg/mL to 500 mg/mL in step (c).

23. The method of claim 17 where the composition is cooled to a temperature in the range of −20° C. to 4° C. in step (d).

24. An elution control matrix for the controlled release of a polypeptide, comprising:
a polymeric matrix and polypeptide microparticles within the polymeric matrix,
wherein the polypeptide microparticles comprise predominantly polypeptide and a crosslinked polymeric component.

25. The elution control matrix of claim 24 wherein the polymeric matrix comprises one or more of the following polymers: poly(n-butyl methacrylate), a polyethylene glycol block copolymer, and/or poly(ethylene-co-vinyl acetate).

26. The elution control matrix of claim 24 wherein the microparticles are present in the matrix in an amount in the range of 30% to 70% by weight solids.

27. The elution control matrix of claim 24 which is in the form of a coating on an implantable medical device.
